(19) United States
(12) Patent Application Publication
(10) Pub. No.: US 2007/0196496 A1
(43) Pub. Date: Aug. 23, 2007

(54) Delivery systems for functional ingredients

(76) Inventors: Michael Farber, Ville St. Laurent (CA); Jonathan Farber, Montreal (CA)

Correspondence Address: STERNE, KESSLER, GOLDSTEIN & FOX P.L.L.C., 1100 NEW YORK AVENUE, N.W., WASHINGTON, DC, 20005, US

(21) Appl. No.: 11/584,748

(22) Filed: Oct. 23, 2006

Related U.S. Application Data
(63) Continuation-in-part of application No. 11/110,848, filed on Apr. 21, 2005.
Application No. 11/110,848 is a division of application No. 10/416,547, filed on Jun. 13, 2003, now Pat. No. 7,067,150.
(60) Provisional application No. 60/372,438, filed on Apr. 16, 2002.

(51) Int. Cl. A61K 31/718; A61K 9/14

(52) U.S. Cl. 424/488; 514/60

(57) ABSTRACT

Oral gel delivery systems are provided that comprise an ingestible matrix within which one or more functional ingredients are substantially uniformly and completely dispersed and in which degradation of the functional ingredient(s) is minimised or eliminated. The matrix of the delivery systems comprises a carbohydrate component that comprises one or more carbohydrates that exhibit good moisture binding and low gelatinisation temperature; a sugar component comprising one or more sugars, sugar syrups and/or sugar alcohols; a hydrocolloid component, and a solvent component comprising one or more polyhydric alcohols. The delivery systems can be formulated to comprise a range of functional ingredients including various drugs and nutritional supplements.

### CROSS REFERENCE TO RELATED APPLICATIONS

This application is a continuation-in-part of U.S. patent application Ser. No. 11/110,848, filed Apr. 21, 2005, which is a divisional application of U.S. patent application Ser. No. 10/416,547, filed Jun. 13, 2003, now issued as U.S. Pat. No. 7,067,150, issued Jun. 27, 2006, which is a national stage of PCT application PCT/CA03/00411, filed Mar. 25, 2003. The aforesaid PCT application claims priority from U.S. Provisional Patent Application Ser. No. 60/372,438, filed Apr. 16, 2002. The contents of all of the aforementioned applications are hereby specifically incorporated by reference in their entirety.

### BACKGROUND OF THE INVENTION

1. Field of the Invention

The present invention pertains to the field of oral delivery systems, in particular to oral delivery systems for functional ingredients.

2. Background Art

### BRIEF SUMMARY OF THE INVENTION

Nutritional and dietary supplements such as multi-vitamins and minerals, botanicals and herbal extracts have grown in popularity, as evidenced by the tremendous growth in the industry involved in their manufacture, production and distribution. Such supplements can be consumed in a variety of ways, the most common being in powder or capsule form.

The consumption of powders suffers from problems such as low solubility or dispersability in water or juice and unpleasant mouthfeel and taste. Many supplements are poorly absorbed into the body and a common approach to this problem is to consume larger doses, which can result in unpleasant side effects including cramping, bloating and flatulence. Thus, a number of different delivery systems have been developed to attempt to improve oral methods of delivering various supplements or active ingredients.

A number of encapsulated formulations have been developed which encapsulate or retain functional ingredients in various glassy, sintered or chewy confectionery-type matrixes. In general, the confectionery serves as a solid continuous matrix for the active ingredient or supplement. The active ingredient is delivered according to the dissolution rate of the confectionery matrix, which confers a solid taste in the mouth. Crushing the confectionery is a solution for the consumer to speed up the release of the active ingredient but this solution may be undesirable as dental problems may arise and/or the release rate of the active ingredient incorporated therein may no longer be optimal. Depending upon the method of manufacturing the confectionery matrix, the active ingredient may suffer from deterioration or damage due to heat and/or mechanical stresses in the manufacturing process. Often, high deterioration rates due to strong processing conditions are compensated for by overdosing of the active ingredient in the confectionery matrix, however, this is a costly method resulting in the wastage of a lot of the active ingredient. The “solid” taste a pressed tablet or glassy matrix may provide in the mouth may also be considered as not very attractive in the context of delivering active ingredients, especially if the product is supposed to be primarily a confectionery.

Liquid-filled boiled sweets are known and may also be used to deliver active ingredients. However, despite the fact the centre is primarily liquid, the whole product has a tendency to melt as one piece in the mouth. The liquid centre does not release from the casing rapidly but rather melts slowly and progressively, thus making a pasty mass.

Powdered sugar filling in a high boiled sweet has also been known for many years in the manufacture of traditional confectioneries such as “Sherbet Lemon” in England. This type of confectionery behaves in the mouth in a way similar to liquid-filled boiled sweets with the casing and filling melting slowly in the mouth and has not been used for delivering active ingredients.

Encapsulation of active ingredients has been described in a number of publications. For example, U.S. Pat. No. 5,897,897 describes the encapsulation of medications, pesticides, vitamins, preservatives or flavouring agents within a glassy matrix consisting of modified starch and a polyhydric alcohol and European Patent EP 0904 784 describes a probiotic preparation with health promoting action comprising bacterial cells, novelose and arabic gum included in a 3-gram proteinic capsule. U.S. Pat. No. 5,648,092 describes pharmaceutical compositions in the form of pleasant-tasting chewable tablets, or chewable coated tablets, which contain at least one rapidly swelling physiologically acceptable gel former plus sugar or sugar substitutes in addition to the pharmaceutically active ingredient sulfacrate.

Similarly, a number of publications describe various means for encapsulating probiotic microorganisms. U.S. Pat. No. 4,396,631, for example, describes a Bifidobacterium-containing confectionery tablet including one or more of substances selected from starch, starch hydrolyzate and protein, while Japanese Patent JP 2893021 describes a boiled sweet enclosing bifidobacteria. The Bifidobacteria are encapsulated with a protective coating film and diluted with a mixture of powdered sugar or sugar alcohol as a filling. Japanese Patent JP 60083535 describes a preparation of candies containing Lactobacilli made by mixing sugar and millet honey, chilling, pulverising and adding activated Lactobacilli powder. Japanese Patent JP 57032221 describes candy tablets containing Bifidus microorganisms made by mixing microorganism powder with fat, adding further raw materials and tabletting. A confectionery composition containing a long-life lacetic bacteria, fats and/or oil, fermented milk powder and saccharide is described in European Patent EP 704164 and German Patent DE 19830528 discloses a multi-layer tablet comprising nutritious substances and microorganisms, which can be stored without cooling.

Gelatine, pectin and other hydrocolloids have been used for some time in the candy industry. Great Britain Patent No. 691,782, for example, describes a method for the manufacture of tablet jellies containing pectin; sweetening agents, such as sucrose, invert syrup and glycerol; a polyvalent metal salt, such as calcium chloride; an edible acid, such as citric acid, and about 15% water. U.S. Pat. No. 4,597,981 describes soft candy compositions comprising 9-82% (by weight) hydrogenated starch hydrolysate, sugars, sugar alcohols, dextrose and gelatine and Bell V. L., Research Disclosure, Vol. 348, No. 085 describes gelatine-free marshmallow compositions comprising corn syrup, gellan gum, sodium citrate, sugar, dextrose, starch and water.

Hydrocolloids have also been employed in the preparation of various delivery systems for functional ingredients. For example, U.S. Pat. No. 6,482,465 describes a “no-cook” method of producing a chewy nougat or health bar confectionery. The confectionery can optionally comprise a “bioaffecting agent,” such as calcium carbonate. The method described in this patent for preparing the nougat comprises combining a saccharide-based component, which is preferably substantially dry, with a hydrated hydrobinding component. The hydrobinding component is a proteinaceous material such as gelatine, or a food-grade gum. Bioaffecting ingredients can be added to the saccharide-based component or the hydrobinding component. The resulting “functionalized confectionery mass” has “sufficient internal cohesivity to be handled without losing its integrity as a mass” and the consistency of “a dough or paste.”

U.S. Pat. No. 6,077,557 describes a gelled dried fruit confectionery product fortified with insoluble calcium of a particular particle size and a method for preparing same. The method described for the preparation of the fortified gelled food product comprises preparing a slurry gel base, which includes nutritive carbohydrate sweeteners, a calcium sequestrant, gelling agent(s), an insoluble calcium phosphate salt, moisture and optionally fat, adding sufficient amounts of an edible organic acidulant to provide a gellable fruit base having a pH ranging from about 3.0 to 5.5 and then moulding the fruit base.

U.S. Pat. No. 4,778,676 (and divisional applications: U.S. Pat. Nos. 4,879,108; 4,882,151; 4,882,152; 4,882,153; 4,882,154; 4,882,155; 4,882,156; 4,882,157; 4,882,158; 4,882,159; and 4,882,160) describes a chewable delivery system for actives that comprises a pre-coated active in a confectionery matrix comprising gelatine, glycerine, a sweetener and water. The process for preparing the confectionery delivery system is based on the preparation of a glycerated-gelatine base, which includes first mixing together gelatine and glycerine in water, together with any optional additional hydrocolloid materials, until uniformity is obtained. One or more sweeteners are added, while mixing is continued, and the active is then mixed in. The temperature at which the delivery system is prepared is not specified. The patent also indicates that pre-coating the active is critical to the success of the invention as this step effectively masks the bitterness and undesirable mouthfeel or texture of the active.

U.S. Pat. No. 5,928,664 also describes a gummy delivery system that is based on a glycerated-gelatine matrix. The glycerated-gelatine matrix is prepared by heating an aqueous solution of gelatine and glycerine to a temperature of about 85-100° C. for a sufficient time to remove from about 10% to 100% of the original water content. The glycerated gelatine matrix is then combined with the other components of the delivery system, including the active. The active is generally added as a solid or as part of a “shearform matrix.” The final product is intended to be chewed for a long enough time to ensure delivery of the active.

United States Patent Application 20020197323 describes a process for preparing a delivery system that involves mixing a carbohydrate component, which can include sugars, starch and/or gelatine, with a humectant component, such as maltitol, lactitol, glycerine or sorbitol, and water. The mixture is heated from about 150° F. (65.6° C.) to about 300° F. (148.9° C.) to form a cooked mixture. Once cooled, an emulsifier system, which includes emulsifiers and fats, is mixed into the cooled mixture to form a delivery base, which is further cooled to a temperature below about 110° F. (43.3° C.) to form a stable solid delivery base. At this point, one or more actives can be mixed into the stable solid delivery system, followed by moulding.

U.S. Pat. No. 4,950,689 also describes a gel confectionery delivery system and a method for the preparation of same. The delivery system comprises pectin and an edible insoluble solid in an amount sufficient to strengthen the internal pectin gel network and to maintain gel integrity during removal from moulds. The pectin gel confectionery delivery system can also includes an active component. The method of preparing the gels comprises combining pectin with water, adjusting the pH to below 4.5, adding sugar and mixing until dissolved. The mixture is then boiled in order to obtain the desired solids content level, followed by another adjustment of pH by the addition of acid. A second mixture comprising insoluble solids and optionally a humectant, such as glycerine, is prepared and added to the first mixture at a temperature of about 100° C. The optional active is preferably added at the end of the processing cycle in order to minimise any degradation of the active due to the elevated temperatures used during the preparation of the delivery system.

U.S. Pat. No. 6,432,442 describes a chewable gelatine matrix for the delivery of actives such as vitamins, minerals, antipyretics, analgesics and expectorants, and a method for the preparation of same. The method involves first hydrating gelatine in water at a temperature of about 100° C., then maintaining the hydrated gelatine at a temperature of about 60° C. to 70° C. An additional hydrocolloid can be included in the matrix by combining the hydrocolloid with sugar and corn syrup in a separate container and heating to temperatures above boiling. The water content of the hydrated hydrocolloid/sugar solids suspension is reduced and the mixture cooled to 90° C., and the gelatine mixture added in. At cooler temperatures, appropriate flavourings, colourings and preservatives, can be added. As a final step, the active materials are added, preferably in coated or encapsulated form to enable survival and stability of the actives during processing. Sugarless formulations are also contemplated in which the sweeteners are replaced in the method described above with, for example, polyhydric alcohols such as sorbitol, xylitol, erythritol and maltitol.

U.S. Pat. No. 6,673,380 describes the preparation of a fortified chewy confectionery delivery system by combining and cooking a mixture of fat, carbohydrate and optionally protein to form a precooked mass, incorporating a fortifying component, such as minerals or vitamins, to the precooked mass and cooling the fortified precooked mass to form a fortified caramel confection. The carbohydrate-fat-protein mixture is heated to a temperature ranging from about 220° F. (104.4° C.) to 270° F. (132.2° C.). The carbohydrate component includes reducing and non-reducing sugars and may further comprise, for example, a sugar alcohol, such as sorbitol, maltitol, mannitol and xylitol.

International Patent Application PCT/US97/20217 (WO 98/20860) describes a process for preparing a chewable delivery system for a pharmacologically active material, which includes heating a hydrocolloid, sugar and water with mixing to produce a uniform mixture. Thereafter, a pharmacologically active material, such as calcium carbonate, is added to the mixture and mixing continued until the active is uniformly dispersed. The mixture is subsequently heated in order to evaporate water from the mixture until a predetermined weight is achieved.

U.S. Pat. No. 5,773,473 describes a creatine dietary supplement comprising creatine solubilised in propylene glycol. The creatine is preferably solubilised in the propylene glycol under high shear. Pharmaceutical compositions comprising the creatine supplement are also mentioned, which may be provided in oral dosage forms such as tablets, dragees, or capsules.

This background information is provided for the purpose of making known information believed by the applicant to be of possible relevance to the present invention. No admission is necessarily intended, nor should be construed, that any of the preceding information constitutes prior art against the present invention.

### SUMMARY OF THE INVENTION

An object of the present invention is to provide an oral delivery system for functional ingredients. In accordance with an aspect of the present invention, there is provided a semi-solid oral gel delivery system for functional ingredients comprising an effective amount of one or more functional ingredients substantially uniformly dispersed throughout a semi-solid matrix, said semi-solid matrix formulated from: (a) between about 8% and about 60% by weight of one or more sugars, sugar syrups, or sugar alcohols, or a combination thereof; (b) between about 0.5% and about 15% by weight of a carbohydrate component comprising one or more starches or modified starches; (c) between about 0.1% to about 15% by weight of one or more hydrocolloids, and (d) between about 5% and about 50% by weight of a solvent component comprising glycerol, wherein said delivery system has a final moisture content of between about 10% and about 40% by weight, a water activity of less than about 0.9, and a melting temperature between about 30° C. and about 60° C.

In accordance with another aspect of the present invention, there is provided a semi-solid oral gel delivery system for functional ingredients comprising an effective amount of one or more functional ingredients substantially uniformly dispersed throughout a semi-solid matrix formulated from between about 8% and about 60% by weight of one or more sugars, sugar syrups, or sugar alcohols, or a combination thereof; between about 0.5% and about 15% by weight of a carbohydrate component comprising one or more starches or modified starches; between about 0.1% to about 15% by weight of one or more hydrocolloids, and between about 5% and about 50% by weight of a solvent component comprising glycerol, wherein said delivery system has a final moisture content of between about 10% and about 40% by weight, a water activity of less than about 0.9, and a melting temperature between about 30° C. and about 60° C. and is prepared by a process comprising the steps of: (a) dispersing said effective amount of the one or more functional ingredients in said solvent component below a temperature of 100° C. to provide a solvent mixture; (b) blending said solvent mixture at a temperature between about 50° C. and 80° C. with a blend comprising said one or more sugars, sugar alcohols or sugar syrups, or combination thereof; said carbohydrate component; said one or more hydrocolloids, and optionally water, to provide a flowable matrix in which said one or more functional ingredients are substantially uniformly dispersed, and (c) moulding said matrix and allowing it to cool to provide said semi-solid oral gel delivery system.

In accordance with one embodiment of the present invention, the semi-solid oral gel delivery system comprises one or more drugs suitable for oral administration are selected from the group of: an acid-lipid agent, an alkaloid, an anabolic drug, an antacid, an anti-asthmatic, an anti-anginal drug, an anti-arrhythmic, an antibiotic, an antibody, an anti-cholesterolemic, an anti-coagulant, an anti-convulsant, an anti-diarrhoeal, an anti-emetic, an anti-fungal, an antigen, an anti-histamine, an anti-hypertensive drug, an anti-inflammatory drug, an anti-manic, an anti-migraine drug, an anti-nauseant, an anti-obesity drug, an anti-psychotic, an anti-pyretic, an anti-spasmodic agent, an anti-thyroid preparation, an anti-thrombotic drug, an anti-tumour compound, an anti-tussive, an anti-uricemic drug, an anti-viral, a cerebral dilator, a cholesterol lowering drug, a contrast agent, a coronary dilator, a decongestant, a diuretic, an erythropoietic drug, an expectorant, a gastrointestinal sedative, a hormone, a hyperglycaemic agent, a hypnotic, a hypoglycaemic agent, a laxative, a local anaesthetic, a mucolytic, a neuromuscular drug, a peripheral vasodilator, a prokinetic drug, a proton pump inhibitor, a psychotropic, a sedative, a stimulant, a thyroid preparation, a tranquilliser, a uterine relaxant, a vasoconstrictor, a vasodilator and a vasopressor.

In accordance with another embodiment of the present invention, the semi-solid oral gel delivery system comprises one or more nutritional supplements are selected from the group of: an antioxidant, an amino acid, an amino acid derivative, a bee product, a botanical extract, a choline source, a co-enzyme, a co-factor, a dipeptide, an enzyme, a fatty acid, a fibre, a herbal extract, a hormone, a joint health nutrient, a macro-nutrient, a metabolic intermediate, a micro-nutrient, a mineral, a mineral salt, an oxygenator, a phospholipid, a phytochemical, a prebiotic, a probiotic bacterium, a protein, and a vitamin.

Various objects and advantages of the present invention will become apparent from the detailed description of the invention.

### BRIEF DESCRIPTION OF THE FIGURES

FIG. 1 demonstrates the enhanced uptake of creatine into the blood following administration of a creatine delivery system prepared according to Example 4 to humans.

FIG. 2 demonstrates serum concentrations of creatine following administration of a delivery system containing varying creatine chelate and/or creatine monohydrate formulations.

FIG. 3 presents dissolution profiles for a calcium delivery system according to one embodiment of the present invention (Formulation A) and a calcium delivery system prepared according to a process described in the art (Formulation B); FIG. 3A depicts the % dissolution expressed as % calcium, and FIG. 3B depicts the % dissolution expressed as % calcium carbonate.

### DETAILED DESCRIPTION OF THE INVENTION

Unless defined otherwise, all technical and scientific terms used herein have the same meaning as commonly understood by one of ordinary skill in the art to which this invention pertains. As used herein, percentage values (%) represent the weight percentages of the total weight of the delivery system unless otherwise specified.

The term “functional ingredient,” as used herein, includes physiologically or pharmacologically active substances intended for use in the treatment, prevention, diagnosis, cure or mitigation of disease or illness, substances intended to improve the general health of an animal and substances that provide some degree of nutritional or therapeutic benefit to an animal when consumed. In one embodiment, the term “functional ingredient” refers to the ISLI European definition that states that a functional food can be regarded as “functional” if it is satisfactorily demonstrated to affect beneficially one or more target functions in the body, beyond adequate nutritional effects in a way that is either an improved state of health and well-being and/or reduction of risk of disease (Scientific Concept of Functional Foods in Europe: Consensus Document, British Journal of Nutrition, Volume 80, supplement 1, August 1998). Non-limiting examples include drugs, botanical extracts, enzymes, hormones, proteins, polypeptides, antigens, nutritional supplements such as fatty acids, antioxidants, vitamins, minerals, as well as other pharmaceutically or therapeutically useful compounds. The functional ingredients may include ingredients having active effects in dental or medical hygiene, bone health, digestive aid, intestinal protection, general nutrition, stress relief, and the like.

The term “drug,” as used herein refers to a pharmacologically active substance that exerts a localised or systemic effect or effects on an animal and/or which is intended for use in the diagnosis, cure, mitigation, treatment, or prevention of disease. The term thus includes therapeutic, prophylacetic and diagnostic substances.

The term “nutritional supplement” as used herein refers to a substance that exerts a physiological effect on an animal and includes substances referred to in the art as “nutraceuticals.” Typically, nutritional supplements fulfil a specific physiological function or promote the health and well-being of the consumer. Examples include but are not limited to, botanical extracts, enzymes, hormones, proteins, polypeptides, antigens, fatty acids, antioxidants, vitamins, minerals, herbs, herbal extracts, amino acids, and the like.

The terms “botanical extract” and “botanical,” as used interchangeably herein, refer to a substance derived from a plant source. Non-limiting examples include echinacea, Siberian ginseng, ginko Biloba, kola nut, goldenseal, goto kola, schizandra, elderberry, St. Johns Wort, valerian and ephedra.

The term “animal” as used herein includes, but is not limited to, mammals (including both humans and non-human mammals), birds and reptiles.

As used herein, the term “about” refers to approximately a +/−10% variation from the stated value. It is to be understood that such a variation is always included in any given value provided herein, whether or not it is specifically referred to.

The Delivery System

The delivery systems according to the present invention are oral gel delivery systems comprising an ingestible matrix within which one or more functional ingredients are substantially uniformly and completely dispersed and in which degradation of the functional ingredient(s) is minimised or eliminated.

The delivery systems according to the present invention are suitable for administration to both human and non-human animals. One skilled in the art will appreciate that each delivery system can be formulated differently according to the type of animal to which it is to be administered. For example, for administration to an animal such as a cat or a dog, meat or fish-based flavours may be added. For administration to a human, the delivery system may be formulated, for example, as a confectionery using fruit-based or other flavours. The oral delivery system of the present invention has texture and density that is analogous to a piece of soft liquorice or a jujube and is especially suited for oral administration due to its palatability. Additionally, due to their highly portable format, the delivery systems are simple and convenient to administer and to consume for both humans and other animals.

The delivery systems of the present invention can be formulated for specific purposes, thus the delivery systems can be formulated to comprise a single functional ingredient or a specific combination of functional ingredients in order to produce a specific physiological effect. A wide variety of such combinations of functional ingredients are known in the art for providing specific physiological benefits and are suitable for inclusion in a delivery system of the invention. Non-limiting, representative examples are provided below in the Section entitled “Administration and Use” and in Table 1.

The delivery systems of the present invention comprise one or more functional ingredients substantially uniformly dispersed within a matrix which comprises 1) a carbohydrate component that comprises one or more carbohydrates that exhibit good moisture binding and low gelatinization temperature; 2) a sugar component comprising one or more sugars, sugar syrups and/or sugar alcohols; 3) a hydrocolloid component, and 4) a solvent component comprising one or more polyhydric alcohols. The matrix may also include one or more sources of monovalent cations or divalent cations if required, for example, to allow for proper set-up of the matrix. If insufficient water is provided by the various components selected to formulate the matrix, additional water may be added to the matrix as necessary to provide the desired final moisture content within the range indicated below. The use of one or more carbohydrates and a hydrocolloid component in amounts within the ranges indicated below results in a matrix that readily retains the solvent component and thereby prevents separation of the solvent from other components of the matrix. Additives such as natural or artificial flavourings, colourings, acidulants, buffers and sweeteners can be included in conventional amounts in the matrix.

In one embodiment, the matrix comprises 1) one or more carbohydrates that exhibit good moisture binding and low gelatinisation temperature; 2) a sugar component comprising one or more sugars, sugar syrups and/or sugar alcohols; 3) a hydrocolloid component; 4) a solvent component comprising one or more polyhydric alcohols; 5) one or more mono- or divalent cations, and 5) water.

The delivery systems may further comprise one or more compounds that act to enhance the bioavailability of one or more of the functional ingredients (i.e. “bioavailability enhancers”), as discussed in more detail below.

Due to the substantially uniform and complete dispersion of the functional ingredients within the matrix, the delivery systems are suitable for division into sub-units. For example, if a single unit of a delivery system of the invention is divided into three subunits, each subunit will contain a third of the dose of the original unit. Such division would not be possible with other delivery systems in which the functional ingredients are not evenly dispersed.

The matrix of the delivery systems provides for minimised degradation of the functional ingredients during the preparation of the matrix and the storage of the final delivery systems. The use of relatively low temperatures in the preparation of the matrix, when compared to typical manufacturing procedures for confectioneries, ensures that the functional ingredients are not degraded by excessive heat. In accordance with the present invention, the delivery systems are prepared at a temperature of 100° C. or less. In one embodiment of the present invention, the delivery systems are prepared at or below a temperature of 75° C. In other embodiments, the delivery systems are prepared at or below a temperature of 70° C., and at or below a temperature of 65° C.

Low temperatures can be employed in the preparation of the delivery system because the matrix is formulated to be flowable at low temperatures by selection of appropriate ingredients as described herein. In one embodiment of the invention, the matrix is formulated to be flowable at or above 45° C. In another embodiment, the matrix is formulated to be flowable at or above 35° C.

In final form, the delivery systems of the present invention are semi-solid, intermediate moisture systems, having a texture similar to soft liquorice or a jujube variety of confectionery. The delivery systems, therefore, are formulated to be semi-solid at normal room temperature. In the event, however, that the delivery system liquefies due to exposure to elevated temperatures, the formulation of the delivery system is such that no phase separation of the components occurs and the delivery system can be readily re-solidified by cooling (for example, by cooling to temperatures of around 4° C.). The reformed product maintains the substantially uniform dispersion of the functional ingredients contained therein. In one embodiment of the present invention, the delivery systems are formulated to be semi-solid at temperatures at or below a temperature of about 45° C., i.e. have a melting point of about 45° C. or higher. In another embodiment, the delivery systems are formulated to be semi-solid at or below about 40° C. In a further embodiment, the delivery systems are semi-solid at or below about 35° C. In other embodiments, the delivery systems are semi-solid at or below about 30° C. and at or below about 25° C.

In a further embodiment, the delivery systems are formulated to have a melting point between about 30° C. and about 60° C., for example between about 35° C. and about 50° C. In another embodiment, the delivery systems are formulated to have a melting point between about 35° C. and about 45° C. In other embodiments, the delivery systems are formulated to have a melting point between about 37° C. and about 45° C., between about 35° C. and about 43° C., and between about 37° C. and about 43° C.

The formulation of the delivery systems is such that flowability of the product intermediates is maintained at each stage in the process of preparing the delivery systems. This provides flexibility in a commercial context, for example, with respect to packaging options as the flowability of the final composition and its ability to be re-liquefied without any substantial phase separation or loss of uniformity of dispersion of the functional ingredients allows the product to be packaged in numerous configurations known in the art using a variety of different packaging processes. Alternatively, in one embodiment, large batches of the delivery system can be prepared and the final product can be held in a bulk storage container or holding tank. The delivery system can then packaged at a later date by melting or re-liquefying the product at a relatively low temperature, which will minimise the input of energy and, when applicable, risk of thermal degradation of the functional ingredient(s), without causing any phase separation of the components or affecting the substantially uniform dispersion of the functional ingredient(s).

The delivery systems also maintain a low interaction with water during and after preparation of the matrix, which contributes to the stability of the functional ingredients dispersed therein. Although the actual amount of moisture and final water activity (a_{w}) of an intermediate moisture food has not been defined precisely in the art, general opinion is that an intermediate moisture product should have a moisture content between about 10% and about 40% by weight and an a_{w} below about 0.9 (see, S. Hegenbart, “Exploring Dimensions in Intermediate Moisture Foods,” (1993) Food Product Design, Weeks Publishing Company, Northbrook, Ill.). In accordance with the present invention, therefore, the final moisture content of the delivery systems is between about 10% and about 40%. In one embodiment of the present invention, the final moisture content of the delivery systems is between about 10% and about 30%. In another embodiment, the final moisture content of the delivery systems is between about 11% and about 25%. In a further embodiment, the final moisture content of the delivery systems is between about 10% and about 15%. In other embodiments, the moisture content is between about 13% and about 20%, between about 14% and about 18%, between about 15% and about 18%, and between about 15% and about 16%.

Furthermore, the delivery systems of the present invention have a low water activity (a_{w}), typically below about 0.9. In one embodiment of the invention, the water activity of the final delivery systems is below about 0.85. In another embodiment, the water activity of the final delivery systems is below about 0.8. In a further embodiment, the water activity is below about 0.7. In another embodiment, the water activity is below about 0.6. Alternatively, the water activity of the final delivery systems may be described as being between about 0.45 and about 0.7. In one embodiment, the water activity is between about 0.5 and about 0.6.

For those functional ingredients that are susceptible to degradation, for example, due to heat liability, degradation during the process of preparing the matrix of the delivery systems is minimised. In accordance with one embodiment of the present invention, degradation of the functional ingredients during the process of preparing the matrix is less than about 20%, i.e. for a given functional ingredient, the amount of the breakdown product(s) of that functional ingredient that is present in a final delivery system is less than 20% of the initial amount of the functional ingredient incorporated into the delivery system. In one embodiment, degradation of the functional ingredients during preparation of the matrix is less than about 15%. In other embodiments, degradation during preparation is less than about 10%, less than about 5%, less than about 2% and less than about 1%.

The matrix also provides for minimised degradation of the functional ingredients dispersed therein during storage of the final delivery systems under normal storage conditions (i.e. at temperatures of 30° C. or below). In accordance with the present invention, therefore, degradation of the functional ingredients during storage of the delivery systems under normal conditions is less than about 20%. In one embodiment, degradation of the functional ingredients during storage is less than about 15%. In other embodiments, degradation during storage is less than about 10%, less than about 5%, less than about 2% and less than about 1%.

The delivery systems of the present invention can be formulated such that the delivery system has a final pH in the range of about 2.5 to about 10.0. In one embodiment, the delivery system has a final pH of between about 2.5 and about 9.5. In another embodiment, the delivery system has a final pH of between about 3.0 and about 9.5. Acidic pH is known in the art to promote degradation of certain functional ingredients. For delivery systems formulated to deliver functional ingredients which are sensitive to, or reactive at, acidic pH, therefore, the final pH of the delivery system is neutral to mildly basic. By neutral to mildly basic pH it is meant that the final pH is between about 6.0 and about 10.0, for example between about 6.0 and about 8.5. In one embodiment of the present invention, the delivery systems are formulated to have a final pH between about 6.2 and about 9.5 and thus are suitable for delivery of functional ingredients that are sensitive to, or reactive at, acidic pH. In other embodiments, the final pH of the delivery systems is between about 6.5 and about 9.5 and between about 7.0 and about 9.5.

For those functional ingredients that are more stable in acidic form, such as trimethylglycine, or functional ingredients which may react with other components at neutral pH such as glucosamine hydrochloride, the pH of the delivery systems may have a final pH below neutral. By below neutral, it is meant that the final pH is between about 2.5 and about 6.8. In one embodiment of the present invention, therefore, the delivery systems are formulated to have a final pH between about 2.5 and about 6.5 and thus are suitable for delivery of functional ingredients that are stable at acidic pH and/or interact with other components at neutral pH. In another embodiment, the delivery systems are formulated to have a final pH between about 2.5 and about 6.0. In a further embodiment, the delivery systems are formulated to have a final pH between about 3.0 and about 6.5. In another embodiment, the delivery systems are formulated to have a final pH between about 3.0 and about 6.3.

It will be readily apparent to one skilled in the art that new formulations of carbohydrate and hydrocolloid or modifications or substitutes thereof are being developed within the food industry. The present invention therefore contemplates the use of such new formulations to prepare the matrix of the present invention provided that the final properties of the delivery systems are maintained, i.e. substantially uniform and complete dispersion of the functional ingredients, minimisation of the degradation of the functional ingredients and a final moisture content for the delivery systems of between about 10% and about 40% and a water activity below about 0.9. For example, a whey-based polymer has recently been developed that acts as a gelling agent (Dairy Management Inc™). The polymer mimics gelatine functionality and forms strong gels at room temperature that exhibit large deformation without fracture and may be suitable for use in the matrix in accordance with the present invention.

The texture, physical attributes, form and shape of the matrix as described below, can be varied by altering the ratio of ingredients within the given ranges using the methods described herein or by methods familiar to a worker skilled in the art.

One skilled in the art will appreciate that specific selections of the possible components provided below, must be safe for animal consumption. Components for inclusion in the delivery systems are, therefore, substances that are generally regarded as safe (GRAS) and/or meet regulatory standards, such as those of the Codex Alimentarus. Examples falling within the general descriptions provided below that are significantly toxic or cause other types of significant harm to animal health are explicitly excluded from the description of the invention.

1. The Matrix

As indicated above, the delivery systems of the invention comprise one or more functional ingredients dispersed in a matrix that comprises 1) a carbohydrate component that comprises one or more carbohydrates that exhibit good moisture binding and low gelatinisation temperature; 2) a sugar component comprising one or more sugars, sugar syrups and/or sugar alcohols; 3) a hydrocolloid component, and 4) a solvent component comprising one or more polyhydric alcohols. One skilled in the art will appreciate that some of these categories of components overlap, for example, sugars are also carbohydrates and some carbohydrates (such as starches and polysaccharide gums) are also hydrocolloids. For greater clarity, therefore, the use of these terms in the context of the present invention is described below with reference to exemplary, non-limiting compounds.

1.1 Carbohydrate Component

The carbohydrate component of the matrix typically performs the functions of water binding and gelation and contributes to the overall texture and body of the final delivery system. The carbohydrate component contributes to the structural integrity of the matrix and its low set temperature. The carbohydrate component can also provide heat stability to the finished product as well as the ability to bind a limited quantity of fats/oils if required.

The one or more carbohydrate(s) to be included as the carbohydrate component of the matrix are selected for their ability to fully hydrate and develop their viscosity in the presence of the other matrix-forming components at a temperature below 100° C. The selected carbohydrate should thus be capable of dispersing without clumping in a sugar syrup and/or in water, and of becoming fully hydrated with or without heating in the presence of a sugar syrup and/or another source of water. While the majority of carbohydrates hydrate upon heating, certain starches, which are commercially available and are known in the art as “cold set” or “pre-gelatinised” starches are capable of hydrating at room temperature and are also suitable for use in the matrix according to the present invention.

In accordance with the present invention, therefore, the selected carbohydrate(s) are capable of hydrating and developing their viscosity at a temperature below 100° C. In one embodiment, the carbohydrate(s) are capable of hydrating at or below about 70° C. In another embodiment, the carbohydrate(s) are capable of hydrating at or below about 50° C. In other embodiments, the carbohydrate(s) are capable of hydrating at or below about 40° C., at or below about 35° C. and at or below about 25° C.

Furthermore, the selected carbohydrate(s) should allow the final matrix to remain in a free-flowing state at a sufficiently low temperature for addition of the functional ingredients without significant degradation of these compounds. In accordance with the present invention, therefore, the carbohydrate remains free-flowing at or below 100° C. In one embodiment of the present invention, the carbohydrate remains free-flowing between about 35° C. and about 85° C. In another embodiment, the carbohydrate remains free-flowing between about 45° C. and about 70° C.

The viscosity development of the selected carbohydrate should allow for sufficient ease of mechanical handling and pumping during production as well as allowing sufficient time to incorporate all the ingredients and to mould the final product before it sets. As is known in the art, some carbohydrates develop their viscosity upon heating, whereas others develop viscosity upon cooling. Both types of carbohydrates are considered to be suitable for use in the matrix of the present invention. In one embodiment, the selected carbohydrate will develop its viscosity upon cooling. In another embodiment, the viscosity of the carbohydrate will develop completely after deposition or filling.

Carbohydrates that meet the above criteria are known in the art. Examples include cellulose (or vegetable) gums, starches and other amylaceous ingredients that have been modified such that they have a low set temperature. An amylaceous ingredient as used herein refers to a food-stuff that contains a preponderance of starch and/or starch-like material. Examples of amylaceous ingredients include cereal grains and meals or flours obtained upon grinding cereal grains such as corn, oats, wheat, milo, barley, rice, as well as the various milling by-products of these cereal grains such as wheat feed flour, wheat middlings, mixed feed, wheat shorts, wheat red dog, oat groats, hominy feed, and other such material. Other sources of amylaceous ingredients include tuberous foodstuffs, such as potatoes, tapioca, and the like.

Suitable starches are typically modified starches and include those derived from a natural source, such as those obtained from various plant species. Examples of plant sources of starch include, but are not limited to, corn, waxy corn, wheat, rice, tapioca, potato, pea and other sources known in the art. Modified starches are known in the art and the term generally refers to starch that has been physically or chemically altered to improve its functional characteristics. Suitable modified starches include, but are not limited to, pre-gelatinised starches, low viscosity starches (such as dextrins, acid-modified starches, oxidized starches and enzyme modified starches), derivatised starches, stabilised starches (such as starch esters and starch ethers), cross-linked starches, and starches that have been submitted to a combination of treatments (such as cross-linking and gelatinization) and mixtures thereof. The carbohydrate may also be a synthetic starch substitute provided that it meets the criteria outlined herein.

Suitable cellulose gums for use in the preparation of the matrix are typically modified cellulose gums. Examples of modified cellulose gums include, for example, methylcellulose (MC), hydroxypropyl methylcellulose (HPMC), ethyl cellulose (EC), hydroxyethyl cellulose (HEC), hydroxypropylcellulose (HPC), hydroxypropyl methylcellulose acetate, hydroxyethyl methylcellulose, hydroxyethylcellulose acetate, hydroxyethyl ethylcellulose and combinations thereof. Such modified celluloses are well known in the food industry, for example, a range of modified celluloses known as Methogel Food Gums are manufactured by Dow Chemical Company. In one embodiment of the present invention, the carbohydrate component used in the preparation of the matrix comprises methylcellulose, hydroxypropyl methylcellulose or a combination thereof.

In one embodiment of the present invention, the carbohydrate component comprises a starch and optionally a cellulose gum. In another embodiment, the carbohydrate component comprises a modified starch. In a further embodiment, the carbohydrate component comprises a modified cornstarch. Various modified starches are available commercially, for example, from A.E. Staley Manufacturing Co. Examples include, but are not limited to, the modified cornstarches Soft-Set® and MiraQuick®. The use of combinations of modified starches and modified celluloses as the carbohydrate component of the matrix is also discussed below in Section 1.7.

In accordance with the present invention, the carbohydrate component of the matrix ranges from about 0.5% to about 15% by weight. In one embodiment, the carbohydrate component of the matrix ranges from about 0.6% to about 15% by weight. The selection of the actual amount of carbohydrate from within this range to be included in the matrix will be dependent upon the type of carbohydrate being used and on desired texture of the final product. Determination of this amount is considered to be within the ordinary skills of a worker in the art.

In one embodiment of the present invention, the carbohydrate component used in the preparation of the matrix includes one or more modified starches, which are included in the matrix in a total amount between about 0.5% and about 12%. In another embodiment, the amount of modified starch(es) included in the matrix is between about 0.5% and about 10% by weight. In a further embodiment, the amount of modified starch(es) included in the matrix is between about 0.5% and about 10% by weight. In another embodiment, the amount of modified starch(es) included in the matrix is between about 0.5% and about 9% by weight.

In an alternative embodiment, the amount of modified starch(es) included in the matrix is between about 2% and 15% by weight, for example, between about 2% and 10% by weight. In other embodiments, the amount of modified starch(es) included in the matrix is between about 2% and about 9% by weight, between about 2% and about 8% by weight, between about 2% and about 5% by weight, and between about 2% and about 4% by weight.

In still another embodiment of the present invention, the carbohydrate component used in the preparation of the matrix includes one or more modified celluloses, which are included in the matrix in a total amount between about 0.6% and about 3% by weight. In another embodiment, the amount of modified cellulose(s) included in the matrix is between about 0.6% and 1.5%.

1.2 Sugar Component

Sugar is generally used in a confection primarily for sweetness; however, it is known in the art that sugar can also play an important role in the physical properties of a matrix, such as crystallinity, gel strength, bodying/texture, humectancy, and water activity.

The sugar component of the matrix comprises one or more sugars, sugar syrups, sugar alcohols and/or sugar alcohol solids. Examples include, but are not limited to, sugars such as sucrose, glucose, xylose, ribose, maltose, galactose, dextrose, and fructose; syrups such as corn syrups, hydrogenated glucose syrups, high fructose corn syrups; polydextrose; and sugar alcohols such as isomalt, maltitol, lactitol and mannitol. The latter are also often in the form of syrups. One skilled in the art will appreciate that if a sugar or sugar alcohol solid is used in the matrix, it should be first dissolved, for example, by heating in water or in another syrup, prior to being added to the mixture.

When the sugar used to prepare the matrix is dextrose, it is generally provided in the form of a corn syrup. Corn syrups are prepared by hydrolysis of starch and are characterised by dextrose equivalent (D.E.) values such that they are classified as low, medium or high D.E. syrups, with high D.E. syrups having a high concentration of dextrose and low D.E. syrups having a low concentration of dextrose. In one embodiment of the present invention, the sugar component used in the preparation of the matrix comprises a corn syrup. In another embodiment, the matrix comprises a corn syrup that exhibits a D.E. of between 20 D.E. and 99 D.E. In other embodiments, the matrix comprises a “high” DE corn syrup with a D.E. of between 40 and 70, or with a D.E. of between 62 and 65. Corn syrups can also be employed as a source of fructose, for example, high fructose corn syrups. In one embodiment, the sugar component used in the preparation of the matrix comprises a high fructose corn syrup.

Various corn syrups are commercially available. For example, 62 D.E. 1600 Corn Syrup (Casco Inc./Canada Starch Operating Co. Inc.), SWEETOSE 4300 corn syrup (a 63 D. E. corn syrup; A. E. Staley Manufacturing Company; Decatur, Ill.) and Clearsweet® 63/43 IX corn syrup (a 63 D. E. corn syrup; Cargill/North America Sweeteners).

Combinations of sugars or sugar syrups are also suitable for use in the preparation of the matrix. Examples of suitable combinations of syrups include, but are not limited to, various combinations of isomalt syrup, high fructose corn syrup, corn syrup and maltitol syrup, such as isomalt syrup and high fructose corn syrup, corn syrup and high fructose corn syrup, and maltitol syrup and high fructose corn syrup.

One skilled in the art will appreciate that the total amount of sugar in the matrix will vary depending upon the combination of sugar sources used. For example, when sugar syrups are used, lower viscosity sugar syrups will produce a matrix with less body and lower rigidity. The total amount of the sugar component present in the matrix is about 10% to about 60% by weight.

In one embodiment of the present invention, the sugar component comprises a mixture of sugar syrups. In another embodiment, the sugar component comprises a mixture of sugar syrups in a total amount of between about 10% to about 60% by weight. In a further embodiment, the sugar component comprises a mixture of sugar syrups in a total amount between about 15% and about 55% by weight of the delivery system. In other embodiments, the sugar component comprises a mixture of sugar syrups in a total amount between about 20% to about 60% by weight, between about 25% and about 55% by weight and between about 35% and about 55% by weight of the delivery system.

1.3 Hydrocolloid Component

The matrix according to the present invention further comprises one or more hydrocolloid. Hydrocolloids are hydrophilic polymers of vegetable, animal, microbial or synthetic origin, and are generally added to foodstuffs for a variety of reasons due to their unique textural, structural and functional properties. For example, hydrocolloids can be used for their thickening and/or gelling properties as well as their water binding and organoleptic properties. Hydrocolloids can also be used to improve and/or stabilise the texture of a food product while inhibiting crystallisation.

Suitable hydrocolloids include non-carbohydrate based hydrocolloids, which are typically animal derived, and carbohydrate-based hydrocolloids, such as polysaccharide gels, which are typically plant derived. A representative example of a non-carbohydrate based hydrocolloid is gelatine (hydrolysed collagen). Examples of polysaccharide gels include, but are not limited to, Konjac, tragacanth gum, guar gum, acacia gum, karaya gum, locust bean gum, xanthan gum, agar, pectin, carageenan, gellan gum, and alginate. The use of hydrocolloids is well-known in the art and many hydrocolloids for use in products for human or animal consumption are available commercially, for example, gelatines from Leiner Davis, various polysaccharide gums and blends including Kelcogel® Gellan Gum from CP Kelco, and a range of Ticagel® hydrocolloids from TIC Gums.

One skilled in the art will appreciate that the selection of the hydrocolloid to be used in the matrix will depend on the pH of the matrix, the interaction of the hydrocolloid with the carbohydrate component of the matrix or, if more than one hydrocolloid is used, the interaction of the hydrocolloids, as well as the particular texture and consistency required for the final product. Certain combinations of hydrocolloids are known in the art to provide synergistic effects, for example, the combination of xanthan (which does not gel well alone) with Konjac, or carageenan and Konjac.

The type of hydrocolloid used will also affect the set temperature of the matrix. For example, the use of a gelatine/gellan mixture or a gelatine/pectin mixture provides a set temperature around 35° C., whereas the use of carageenan or locust bean gum will result in a set temperature closer to 60° C. Thus, the choice of hydrocolloid for use in the matrix is also dependent upon the properties of the functional ingredient(s) to be incorporated into the delivery system. Functional ingredients that are unstable at higher temperatures will require the selection of a hydrocolloid or mixture of hydrocolloids that have a low set temperature, whereas functional ingredients that are more stable can be used with hydrocolloids having a higher set temperature. Selection of an appropriate hydrocolloid is considered to be within the ordinary skills of a worker in the art.

In one embodiment of the present invention, the matrix comprises gelatine. The term “gelatine” refers to a heterogeneous mixture of water-soluble proteins of high average molecular weight derived from the collagen-containing parts of animals, such as skin, bone and ossein by hydrolytic action, usually either acid hydrolysis or alkaline hydrolysis. Different types of gelatine can be prepared by altering the process parameters. Gelatine is defined generally using a “Bloom value” which indicates the strength of the gel formed under certain circumstances using the gelatine. In the preparation of confectionery, when a harder gel is desired, gelatine having a higher Bloom value is used. Conversely, when the final product is required to be more flowing, gelatine having a lower Bloom value is used. One skilled in the art will appreciate that the water holding capacity of gelatine alone is lower than that of a combination of gelatine with another hydrocolloid, such as gellan or pectin, and may necessitate the use of a higher amount of gelatine to achieve the desired gelation/texture of the matrix. When the hydrocolloid in the matrix of the present invention comprises gelatine, the Bloom value (BL) is generally about 100 to 260 BL. In one embodiment, the Bloom value is about 250 BL. In another embodiment, a mixture of gelatines with different Bloom values is used.

As indicated above, gelatine can be combined with one or more other hydrocolloids to impart slightly different characteristics to the matrix. For example, combinations of gelatine with gellan or gelatine with pectin provide a good texture to the matrix. When combinations of gelatine and gellan are used in the preparation of the matrix, the ratio of gelatine:gellan is typically in the range between about 15:1 to about 40:1. These relative amounts provide a cohesive structure to the delivery system. When a combination of gelatine and pectin are used in the preparation of the matrix, the ratio of gelatine:pectin is typically in the range between about 15:1 to about 40:1.

In one embodiment of the present invention, a combination of gelatine and gellan is used in the preparation of the matrix in a gelatine:gellan ratio of about 15:1 to about 35:1. In another embodiment, a combination of gelatine and pectin is used in the preparation of the matrix in a gelatine:pectin ratio of about 15:1 to about 25:1.

The total amount of hydrocolloid incorporated into the matrix is generally between about 0.1% and about 15% by weight, for example between about 0.1% and about 12%. In one embodiment, the amount of hydrocolloid incorporated into the matrix is between about 0.5% and about 12% by weight. In another embodiment, the amount of hydrocolloid incorporated into the matrix is between about 1.0% and about 12% by weight. In a further embodiment, the amount of hydrocolloid incorporated into the matrix is between about 2.0% and about 12% by weight.

In an alternative embodiment, the amount of hydrocolloid incorporated into the matrix is between about 0.1% and about 7.0% by weight. In other embodiments, the total amount of hydrocolloid in the matrix is between about 0.5% and about 6.8% by weight, between about 1.0% and about 6.6%, between about 2.0% and about 6.0%, between about 4.0% and about 6.0%, and between about 4.0% and about 7.0%.

1.4 Solvent Component

The primary role of the solvent component of the matrix is to dissolve or disperse the one or more functional ingredients to allow for substantially uniform and complete incorporation of these ingredients into the matrix. In some embodiments, the solvent can also provide body and/or texture to the matrix, improved flow characteristics and/or can function somewhat as a humectant. In accordance with the present invention, at least one functional ingredient is added to the solvent component prior to combining with the remaining components of the matrix.

The solvent used in the preparation of the matrix is typically colourless, non-volatile with no strong odour or flavour and is substantially miscible with water and/or alcohols. In accordance with the present invention, the solvent component comprises one or more polyhydric alcohol. The term “polyhydric” as used herein means that the compound contains two or more hydroxyl groups. Examples of polyhydric alcohols include, but are not limited to, glycerol and/or its lower alkyl ester derivatives, propylene glycol, and short chain polyalkylene glycols, such as polyethylene glycol, and mixtures thereof. As will be apparent to one skilled in the art, certain polyhydric alcohols may also function somewhat as sweeteners.

In one embodiment of the present invention, the solvent component comprises glycerol. In another embodiment, the solvent component comprises glycerol and a short chain polyalkylene glycol. In another embodiment, the solvent component comprises glycerol and polyethylene glycol. In a further embodiment, the solvent component comprises glycerol and propylene glycol.

Typically, the delivery system according to the present invention contains about 5% to about 50% by weight of the solvent component, for example between about 5% and about 48%. Utilising an amount of solvent towards the higher end of this range in the matrix can impart increased mouth-melting properties to the final delivery system allowing, for example, the product to dissolve more rapidly in the mouth. For example, when preparing a delivery system for the functional ingredient calcium, the use of an amount of solvent towards the higher end of this range can improve the texture of the final product. In general, when higher amounts of solvent are employed in the preparation of the matrix, the amount of sugar component included in the matrix is decreased accordingly.

In one embodiment, the delivery system contains about 5% to about 35% by weight of the solvent component. In an alternate embodiment, the delivery system contains about 10% to about 50% by weight of the solvent component. In a further embodiment, the delivery system contains about 20% to about 48% by weight of the solvent component. In other embodiments, the delivery system contains between about 15% and about 50%, between about 15% and about 40% and between about 15% and 35% by weight of the solvent component.

1.5 Mono- or Divalent Cations

If necessary, the matrix can also comprise one or more sources of monovalent cations and/or divalent cations to facilitate gelation of the matrix. Suitable sources of mono- and divalent cations for incorporation into food products are known in the art and are commercially available. Non-limiting examples include mono- or divalent salts, such as sodium chloride, potassium chloride, calcium chloride and potassium citrate. Mono- or divalent salts can be added to the matrix, if required, in an amount between, for example, about 1% and about 5% by weight. In one embodiment, mono- or divalent salts can be added in an amount between about 1% and about 3% by weight. In another embodiment, mono- or divalent salts can be added in an amount between about 1.2% and about 2.5% by weight.

1.6 Water

As indicated above, the delivery system according to the present invention has a final moisture content between about 10% and about 40% and a water activity below about 0.9. In one embodiment, the final moisture content of the delivery system is between about 10% and about 30% and the water activity is below about 0.7. It will be readily apparent to one skilled in the art that the appropriate amount of water may be provided by one or more of the various components of the system, for example, a sugar syrup, a hydrated starch or a hydrated hydrocolloid. Alternatively, when the components of the matrix do not supply sufficient water to provide the delivery system with a final moisture content within the above-noted range, then additional water can be added separately. This additional water can be provided alone or as a solution containing other additives, for example, as a buffer solution or as a solution containing a sweetener, flavouring or colouring. The total amount of water from the one or more sources will be sufficient to provide the final delivery system with a moisture content and water activity within the ranges indicated above.

1.7 Other Additives

The gel matrix can optionally contain other additives such as additional sweeteners, flavourings, colourings, modified vegetable gums or celluloses, or a combination thereof. It will be readily apparent that additives for inclusion in the matrix should be selected such that they do not affect the properties of the matrix, do not exhibit substantial reactivity with the functional ingredients in the matrix, and are stable during preparation of the matrix.

One or more additional sweeteners can be selected from a wide variety of suitable materials known in the art. Representative, but non-limiting, examples of sweeteners include xylose, ribose, sucrose, mannose, galactose, fructose, dextrose, maltose, lactose, maltodextrins, and mixtures thereof. In addition to these sweeteners, polyhydric alcohols such as sorbitol, mannitol, xylitol, and the like may also be incorporated. Alternatively, one or more artificial sweeteners or a blend of artificial sweeteners can be used, for example, sucrose derivatives (such as Sucralose), amino acid based sweeteners, dipeptide sweeteners, saccharin and salts thereof, acesulfame salts (such as acesulfame potassium), cyclamates, steviosides (for example, stevia), dihydrochalcone compounds, thaumatin (talin), glycyrrhizin, aspartame, neotame, alitame, and mixtures thereof. In one embodiment of the invention, the matrix comprises one or more additional sweeteners. In another embodiment, the matrix comprises one or more artificial sweeteners.

When an additional sweetener is used, it can be used in amounts as low as 0.01% by weight. The actual amount of sweetener required will be dependent on the type of sweetener selected and on the desired sweetness of the final product. Amounts of various sweeteners to be added to food products are well known in the art. The total amount of the sugar component, which forms a structural part of the matrix, and additional sweetener(s) in the matrix, however, remains less than 60% by weight.

Suitable flavourings that can be added to the delivery system are known in the art and include, both synthetic flavour oils and oils derived from various sources, such as plants, leaves, flowers, fruits, nuts, and the like. Representative flavour oils include spearmint oil, peppermint oil, cinnamon oil, and oil of wintergreen (methylsalicylate). Other useful oils include, for example, artificial, natural or synthetic fruit flavours such as citrus oils including lemon, orange, grape, lime and grapefruit, and fruit essences including apple, strawberry, cherry, pineapple, banana, raspberry and others that are familiar to a worker skilled in the art. A wide variety of synthetic flavourings suitable for inclusion in the matrix are known in the art and are commercially available. The amount of flavouring agent employed is normally a matter of preference subject to such factors as concentration/dilution of the flavour stock, flavour type, base type and strength desired. In general, amounts of about 0.01% to about 5.0% by weight of a final product are useful. In one embodiment of the present invention, a flavouring agent is included in the matrix in amounts of about 0.02% to about 3%. In another embodiment, the flavouring agent is added in amounts of about 0.03% to about 1.5%.

Colourings suitable for use in foodstuffs are well known in the art and can be optionally included in the matrix to add aesthetic appeal. A wide variety of suitable food colourings are available commercially, for example, from Warner Jenkins, St. Louis, Mo. Where a synthetic colouring agent is used in the matrix, the amount ranges from about 0.01% to about 2% by weight. In one embodiment of the present invention, a synthetic colouring agent is added to the matrix in an amount between about 0.03% to about 1% by weight. A worker skilled in the art will appreciate that when a colouring agent derived from a natural source is used in the matrix, an increased amount of the colouring agent is generally required to achieve the same effect as a synthetic colouring agent.

The present invention also contemplates that modified vegetable gums or modified or unmodified celluloses may be included in the matrix in order to improve the texture, body, lubricity and/or elasticity of the matrix. For example, when the carbohydrate component of the matrix comprises a modified starch, a modified vegetable gum or cellulose may be included. These compounds can be used, for example, to increase the viscosity of the delivery system if it is warmed, thus reducing potential melting and lessening water activity which will help to improve the stability of the system in the event it is left in an excessively hot environment. Examples of modified vegetable gums or modified celluloses are provided above. Unmodified celluloses are also contemplated and are known in the art. Examples include Solka-Floc® (International Fibre Corporation, North Tonawanda, N.Y.) and powdered Avicel® microcrystalline cellulose (FMC Biopolymers, Philadelphia, Pa.). Modified vegetable gums can be included in the matrix in amounts between about 0.01% and 2.0% by weight, for example, between about 0.1% and about 1.5%. Modified or unmodified celluloses, or mixtures thereof, can be included in the matrix in amounts between about 0.1% and about 10.0% by weight, for example, between about 0.6% and about 5.0%.

2. Functional Ingredients

The delivery systems according to the present invention comprise one or more functional ingredients. The functional ingredients to be incorporated into the delivery system can be drugs (i.e. therapeutic and/or diagnostic compounds), nutritional supplements that fulfil a specific physiologic function or promote the health an/or well-being of the consumer, botanicals or herbal extracts, and the like that are suitable for oral administration.

A variety of orally administered drugs are suitable for use with the present delivery system. Representative examples include, but are not limited to:
Alkaloids, such as codeine phosphate and codeine sulfate;
Antacids, such as aluminium hydroxide, calcium carbonate, magnesium hydroxide, and magnesium trisilicate;
Anti-anginal drugs, such as erthyrityl tetranitrate, isosorbide mononitrate and nitroglycerin;
Anti-arrhythmics such as N-acetyl-procainamide;
Antibiotics, such as penicillin, tetracylines, and fluoroquinolones;
Anti-cholesterolemic and acid-lipid agents such as gemfibrozil;
Anti-diarrhoeals, such as glycopyrrolate and loperamide hydrochloride;
Anti-emetics, such as dimenhydrinate, dronabinol, ondansetron hydrochloride;
Anti-fungals, such as fluconazole, ketoconazole, griseofulvin and terbinafine;
Anti-histamines, such as chlorpheniramine maleate, phenindamine tartrate, pyrilamine maleate, doxylamine succinate, and phenyltoloxamine citrate;
Anti-inflammatory drugs, including NSAIDs (such as salicylic acid and its derivatives (for example, aspirin (acetyl salicylic acid)), acetaminophen, and ibuprofen), and steroids (such as prednisone);
Anti-migraine drugs, such as ergotamine tartrate;
Anti-pyretics such as acetaminophen, aspirin and ibuprofen;
Anti-spasmodic agents, such as dicyclomine and scopolamine;
Anti-tumour compounds, such as tacrolimus hydrate, and capecitabine;
Anti-tussives, such as dextromethorphan, dextromethorphan hydrobromide, noscapine, carbetapentane citrate, and chlophedianol hydrochloride;
Anti-virals, such as acyclovir, valacyclovir, ddI and ddA;
Decongestants, such as phenylephrine hydrochloride, phenylpropanolamine hydrochloride, pseudoephedrine, hydrochloride ephedrine;
Expectorants such as guaifenesin;
Laxatives, such as bisacodyl, casanthrol and phenolphtalein;
Local anesthetics, such as lidocaine, benzocaine and oxethazaine;
Prokinetic drugs, such as bethanechol chloride, cisapride and metoclopramide hydrochloride;
Proton pump inhibitors, such as omeprazole, lansoprazole, pantoprazole, rabeprazole and esomeprazole; and
Vasopressors, such as digitoxin.

Other examples of suitable drug categories include, for example, anabolic drugs; anti-asthmatics; anti-coagulants; anti-convulsants; anti-hypertensive drugs; anti-manics; anti-nauseants; anti-obesity drugs; anti-psychotics; anti-spasmodics; anti-thrombotic drugs; anti-uricemic drugs; cerebral dilators; cholesterol lowering drugs; coronary dilators; diuretics; erythropoietic drugs; gastrointestinal sedatives; hyper- and hypoglycaemic agents; hypnotics; mucolytics; neuromuscular drugs; peripheral vasodilators; psychotropics; sedatives; stimulants; thyroid and anti-thyroid preparations; tranquillisers; uterine relaxants; vasoconstrictors and vasodilators; as well as hormones, antibodies, antigens and other bioagents; and contrast agents for medical diagnostic imaging.

One or more of the functional ingredients included in the delivery system can be a nutritional supplement. Illustrative, but non-limiting, examples of nutritional supplements suitable for use with the delivery system according to the present invention include, probiotic bacteria, prebiotics, vitamins, enzymes, co-enzymes, cofactors, antioxidants, minerals, mineral salts, amino-acids, amino acid derivatives (for example, dimethylglycine), peptides, proteins, gums, nutritional carbohydrates, phytochemicals, dextroses, phospholipids, other trace nutrients, oxygenators, brain-stimulating substances, energy providers, metabolic intermediates, hormones, enzymes, botanical extracts, fatty acids (for example, linoleic acid and conjugated linoleic acid), choline sources (for example, lecithin, glyceryl phosphorylcholine and phosphatidylserine), oat beta-glucan or other functional fibres, or combinations thereof. One skilled in the art will appreciate that some of the above categories overlap and are not intended to be mutually exclusive.

Probiotic microorganisms in the form of live microbial nutritional supplements and which are recognized as conferring a beneficial effect on an animal can be incorporated into the delivery system. Probiotic microorganisms are microorganisms which beneficially affect a host by improving its intestinal microbial balance (see, for example, Fuller, R; 1989; J. Applied Bacteriology, 66: 365-378). Beneficial effects of probiotic microorganisms include activation of the immune system, prevention of the bacterial overgrowth by pathogens, prevention of diarrhoea and/or restoration of intestinal flora. Examples of probiotic microorganisms include, but are not limited to, Bifidobacterium (such as Bifidobacterium longum B129, Bifidobacterium longum B128, Bifidobacterium adolescentis Bad4, and Bifidobacterium lactis Bbl2), Lactobacillus (such as, Lactobacillus johnsonii and Lactobacillus paracasei), Streptococcus and Saccharomyces. Typically, the microorganism is added to the matrix in a spray dried or freeze-dried form.

Many probiotic bacterial strains have been deposited under the Budapest Treaty at the Collection Nationale de Cultures de Microorganismes (CNCM), Institut Pasteur, 28 rue du Docteur Roux, 75724 Paris Cedex 15, France. For example, Lactobacillus johnsonii (NCC 533) has been deposited on 30 Jun. 1992 under reference CNCM 1-1225, Lactobacillus paracasei (NCC 2461) has been deposited on 12 Jan. 1999 under reference CNMC I-2116, Bifidobacterium longum (B129) (NCC490) has been deposited on 15Mar. 1999 under reference CNCM I-2170, Bifidobacterium longum (B128) (NCC481) has been deposited on 15 Mar. 1999 under reference CNCM I-2169, and Bifidobacterium adolescentis (Bad4) (NCC251) has been deposited on 15Mar. 1999 under CNCM I-2168. Bifidobacterium lactis (Bbl2) may be obtained at Hanzen A/S, 10-12 Boege Alle, P.O. Box 407, DK-2970.

The amount of probiotic incorporated into the delivery system will vary according to the specific needs. Typically, the amount of lacetic acid bacteria in one unit of the delivery system is between 10² and 10¹² count/gram, for example, between 10⁷ and 10¹¹ count/gram, or between 10⁸ and 10¹⁰ count/gram.

Prebiotics can be delivered alone or in combination with probiotic bacteria in the delivery system. Prebiotics comprise carbohydrates, generally oligosaccharides, and have the ability to resist hydrolysis by enzymes in the animal digestive tract and thus can reach the colon undegraded to provide a carbohydrate substance particularly suited to growth of probiotic bacteria. Oligosaccharides may be produced from glucose, galactose, xylose, maltose, sucrose, lactose, starch, xylan, hemicellulose, inulin, or a mixture thereof. Purified commercially available products such as fructooligosaccharide contain greater than about 95% solids in the form of oligosaccharides. Prebiotics often comprise a mixture of fructooligosaccharide and inulin, for example, PREBI01® or a mixture of commercially available RAFTILOSE® and RAFTILINE® commercialized by Orafti. A prebiotic of this kind has been demonstrated to improve the response of the immune system.

Other suitable nutritional supplements include vitamins and minerals that the body is usually not capable of synthesizing and which are necessary for ensuring normal growth and/or daily body maintenance. In the context of the present invention, the vitamins can be hydrosoluble or liposoluble vitamins. Examples includes, but are not limited to, Vitamin A (axerophtol or retinol), Vitamin D, Vitamin E (alpha-tocopherol), Vitamin K, Vitamin C (L-ascorbic acid), and the B-complex vitamins (thiamine (B₁), riboflavin (B₂), niacin (B₃), pyridoxine (B₆), folic acid (B₉), cyanocobalamin or methylcobalamin (B₁₂), pantothenic acid and biotin). The dosage of vitamins in the delivery system can be adapted to specific needs. In general, one unit of the delivery system may contain a fraction of the recommended daily amount (RDA) of the desired vitamin. For example, assuming a daily consumption of five units of the delivery system, and following European RDA recommendations, Vitamin A can be used up to 160 μg typically between 70 μg and 90 μg a single unit; Vitamin C up to 12 mg typically between 5 mg and 7 mg a single unit; Vitamin E up to 2 mg typically between 0.8 mg and 1.2 mg a single unit; Vitamin D up to 1 μg typically between 0.4 μg and 0.6 μg a single unit; Vitamin B. up to 0.28 mg typically between 0.12 mg and 0.15 mg a single unit.

Antioxidants can be delivered using the delivery system of the present invention, alone or in combination with other functional ingredients. Examples of antioxidants include, but are not limited to, glutathione, peroxidase, superoxide dismutase, catalase, co-enzyme Q10, honey tocopherols and other tocopherols, lycopenes, beta-carotene or other carotenoids, quertin, rutin, flavonoids, catechins, anthocyanins, eleutherosides and ginsenosides. Some of these antioxidants may be found in significant amounts in plant extracts. Examples include Ginko Biloba leaves that contain Gingko flavonoids, blueberry fruits that contains anthocyanins, Ginseng roots which contain ginsenosides, Eleutherococcus roots which contains eleutherosides. The functional ingredient may also be a phytochemical such as polyphenol, procyanidin, phenolic acid, catechin or epicatechin, isoflavone, terpene or other phytonutritive plant material.

Suitable minerals include macro-nutrients such as sodium, potassium, calcium, magnesium, phosphorus or oligo-elements such as iron, zinc, copper, selenium, chromium, iodine, boron, manganese, or a combination thereof. Macro-nutrients are known to play an essential role in complex metabolisms of the body such as in cellular cation exchange, for example, calcium is an essential constituent of the skeleton. Following EU RDA recommendations and assuming, for instance, an average daily consumption of 5 units of the delivery system. Calcium may be used in amounts of up to 160 mg, typically between 60 mg and 90 mg in a single unit.

Trace elements (or micro-nutrients) are minerals present in the human body in quantity of usually less than 5 g. An example of a trace element is zinc, which has antioxidant properties, helps in the synthesis of metallothionein, is an essential factor for protein synthesis and helps improve the function of the immune system. Following EU RDA recommendations and assuming a daily consumption of 5 units of the delivery system, zinc may be used in amounts of up to 3 mg per unit, typically between 1.3 mg and 1.7 mg.

Selenium is also an antioxidant and is a co-factor for glutathione peroxidase. Selenium is known to contribute to the integrity of muscles and sperm and also plays a role in hepatic metabolism. Selenium deficiencies may lead to sever cardiac, bone or neuromuscular damage. For example, following the European RDA recommendations and assuming a daily consumption of 5 units of the delivery system, Selenium may be used in amounts of up to 11 μg per unit, typically between 4 μg and 6 μg in humans.

Other nutritional supplements include amino acids, di-peptides, polypeptides, proteins or essential fatty acids. Examples of suitable amino acids include glutamine, which provides fuel to gastro-intestinal and immune cells, reduces bacterial translocation and helps prevent muscle loss and improves nitrogen balance, and cysteine, which is known to aid in defense against oxidative stress and in protein synthesis. Other examples of suitable amino acids include the essential amino acids isoleucine, leucine, lysine, methionine, phenylalanine, threonine, tryptophan, valine, arginine and histidine; the non-essential amino acids alanine, asparagine, aspartate, glutamate, glycine, proline and serine; and the non-standard amino acids selenomethionine, taurine, GABA, dopamine, lanthionine, 2-aminobutyric acid, dehydroalanine, ornithine, citrulline and hydroxyproline. Various combinations of these amino acids may also be used. Derivatives of cysteine, such as acetylcysteine and cysteine methionine, are also known to aid in defense against oxidative stress and in protein synthesis and are suitable for incorporation into the delivery systems.

Examples of peptides include the glycopeptides of lacetic origin active in inhibiting the adhesion of the bacteria responsible for dental plaque and caries. More particularly, dental and anti-plaque caries agents of this type comprise active principle(s) selected from kappa-caseino-glycopeptides and deacylated derivatives thereof (also known as “CGMP”). Such active principles have an effectiveness on the dental plaque only after a few seconds in the mouth (see, for example, European Patent Number EP283675). Other peptides include phosphopeptides or salts thereof having anticarie properties such as those having from 5 to 30 amino acids including the sequence A-B-C-D-E where, A, B, C, D and E being independently phosphoserine, phosphothreonine, phosphotyrosine, phosphohistidine, glutamate and aspartate and compositions particularly compositions to teeth including same (see, for example, U.S. Pat. No. 5,015,628).

Proteins suitable for inclusion in the delivery systems include animal derived and plant derived proteins, for example, soy protein, whey protein, casein protein, egg protein and the like.

Other nutritional supplements include creatine, caffeine, a bee product (such as bee pollen, Royal jelly, and bee propolis), chitosan, chondroitin, functional fibres, phospholipids, enzymes known to aid digestion (such as papain, bromelain and lipases), shark cartilage extracts, glucosamine, methylsulfonylmethane (MSM), pregnenolone, Brewer's yeast, blue green algae, camitine, bicarbonates, citrates, fibre, and the like.

The nutritional supplement can be a botanical extract, such as guarana, Gingko biloba, kola nut, goldenseal, Goto kola, schizandra, elderberry, St. John's Wort, valerian, ephedra and ephedra alkaloids, evening primrose oil, beta-sitosterol, cafestol, D-limonene, kahweol, nomilin, oltipraz, sulphoraphane, tangeretin, black tea, white tea, java tea, garlic oil, jojoba, bitter melon, green tea extract, lemon oil, mace, liquorice, menthol, onion oil, orange oil, rosemary extract, milk thistle extract, Echinacea, Siberian ginseng or Panax ginseng, lemon balm, Kava Kava, Yerba Mate, bilberry, soy, grapefruit, seaweed, hawthorn, lime blossom, sage, clove, basil, curcumin, wild oat herb, dandelion, gentian, aloe vera, hops, cinnamon, peppermint, grape, chamomile, fennel, marshmallow, ginger, slippery elm, cardamon, coriander, anise, thyme, rehmannia, eucalyptus, menthol, Citrus aurantium and schisandra.

Isoflavones have also been reported to contribute to bone health and can be included in the delivery systems of the invention. Suitable isoflavones include, but are not limited to, naturally occurring soy isoflavones such as daidzein (4′,7-dihydroxyisoflavone), genistein (4′,5,7-trihydroxyisoflavone), and glycitein, which occur in a variety of forms (for example, in glycosidic and acetylated forms). Soy isoflavones are commercially available, for example, from Archer Daniels Midland (Decatur, Ill.). Synthetically derived isoflavones, such as ipriflavone (a synthetic 7-isopropoxyisoflavone) can also be used.

The delivery systems can comprise up to about 40% by weight of the one or more functional ingredients. In one embodiment, the delivery systems comprise between about 0.01% and about 40% by weight of the one or more functional ingredients. In another embodiment, the delivery systems comprise between about 0.1% and about 40% by weight of the one or more functional ingredients. In a further embodiment, the delivery systems comprise between about 0.2% and about 40% by weight of the one or more functional ingredients.

One skilled in the art will appreciate that the amount of functional ingredient(s) to be incorporated will be dependent on the type of functional ingredient(s) and the requirements of the target consumer. For example, the recommended dosage of a drug or a micro-nutrient, such as a vitamin, is generally less, on a weight by weight basis, than the recommended dosage of a macro-nutrient, such as calcium, or nutritional supplements such as creatine, protein or fibre, which are known to be required in higher amounts in order to provide a physiological effect.

Thus, in one embodiment of the present invention, the total amount of functional ingredients constitute less than about 25% by weight of a delivery system. In another embodiment, the delivery systems incorporate between about 0.01% and about 20% by weight of the functional ingredient(s). In another embodiment, the delivery systems incorporate between about 0.01% and about 15% by weight of the functional ingredient(s). In another embodiment, the delivery systems incorporate between about 0.01% and about 10% by weight of the functional ingredient(s).

In an alternative embodiment, the total amount of the functional ingredient(s) constitutes between about 5% and about 40% by weight of the delivery system. In another embodiment, the total amount of the functional ingredient(s) constitutes between about 7% and about 40% by weight of the delivery system. In a further embodiment, the total amount of the functional ingredient(s) constitutes between about 10% and about 40% by weight of the delivery system.

Selection of appropriate functional ingredients for incorporation into the delivery systems for administration to a given animal is considered to be within the ordinary skills of a worker in the art and it is understood that functional agents suitable for administration to humans may differ from those suitable for other animals. Furthermore, it will be apparent that inappropriate combinations of functional agents, for example, those that interact with each other, should not be included in a delivery system.

As indicated above, the present invention provides for delivery systems containing specific combinations of functional ingredients. A wide variety of such combinations of functional ingredients are known in the art for providing specific physiological or pharmaceutical benefits and are suitable for inclusion in a delivery system of the invention. Non-limiting examples are provided in Table 1.

TABLE 1
Representative examples of types of delivery systems and suggested functional ingredients
for incorporation therein

| Formulation | Suggested Functional Ingredients¹ |
|---|---|
| Energy formulation | Ginseng, chromium picolinate, chromium |
| | chelate, Rhodiola crenulata. |
| Weight loss formulation | Caffeine, ephedra, conjugated linoleic acids (CLA). |
| Thermogenic formulation | Caffeine, tocopherols, Citrus aurantium, |
| | ephedra alkaloids. |
| Memory enhancement | Ginkgo biloba, goto kola. |
| Sexual health | Yohimbe, Kubu pepper. |
| Antioxidant | Vitamin E, vitamin C, Alpha Lipoic Acid |
| | (ALA). |
| Bone health | Calcium, magnesium, vitamin C. |
| Joint health | Methylsulphonylmethane (MSM), |
| | glucosamine, chondroitin. |
| Cold prevention | Echinacea, zinc, vitamin C. |
| Vitamin and/or mineral supplements | B-Vitamin complex, D vitamins, Vitamin |
| (particularly formulations for children) | C, Vitamin co-factors. |
| Dietary supplements | Essential fatty acids, amino acids. |
| Muscle enhancement | Creatine, dimethylglycine, pregnenolone, |
| | amino acids. |
| Sports nutrition | Dehydroepieandrosterone (DHEA), |
| | pregnenolone. |
| Probiotics | Acidiphilus, Bifidus, prebiotics. |
| Digestive aids | Bromelain, papain, lipases, probiotics. |
| Anti-aging formulations | Omega-3 fatty acids, lignan, S-adenosyl |
| | methionine(SAMe), melatonin. |
| Seniors formulations | Calicum, omega-3 fatty acids, SAMe. |
| Women's health | Soy isoflavanones. |
| Cardiovascular health | Arginine, Siberian Ginseng, Vitamin B6, |
| | CoQ10, Rhodiola crenulata. |

¹Delivery systems may contain one, or a combination, of the listed functional ingredients.

The process of preparing the delivery systems of the present invention, as described in more detail below, demonstrates considerable flexibility and broad applicability as can be seen from the range and diversity of exemplary nutritional supplements that have been incorporated into the delivery system either alone or in various combinations (see Table 12; Example 16 below). These nutritional supplements include compounds such as simple mineral salts, including calcium carbonate; simple acids, such as ascorbic acid; fatty acids, such as conjugated linoleic acid; alcohols, such as octacosanol; and more complex structures, such as the carotenoid astaxanthin and the porphyrin vitamin B₁₂, as well as complex mixtures of compounds, such as those found in botanical extracts (for example, ephedra or yerba mate), and thus represent compounds having very different chemical and physical properties. For example, highly water-soluble compounds, such as arginine, creatine, histidine, lysine, and vitamin B₁₂; lipophilic and sparingly water-soluble compounds, such as astaxanthin, β-carotene, conjugated linoleic acid, inulin and Vitamin E; essentially water-insoluble compounds, such as calcium carbonate and octacosanol, and liposomally formulated compounds such as co-enzyme Q₁₀.

3. Bioavailability Enhancers

The present invention also contemplates the optional inclusion of bioavailability enhancers in the delivery systems. Such compounds are known in the art and act to increase the absorption of functional ingredients by the body. Bioavailability enhancers can be natural or synthetic compounds. In one embodiment, the delivery system comprises one or more bioavailability enhancers in order to enhance the bioavailability of the functional ingredient(s).

Natural bioavailability enhancers include ginger, caraway extracts, pepper extracts and chitosan. The active compounds in ginger include 6-gingerol and 6-shogoal. Caraway oil can also be used as a bioavailability enhancer (U.S. Patent Application 2003/022838). Piperine is a compound derived from pepper (Piper nigrum or Piper longum) that acts as a bioavailability enhancer (see U.S. Pat. No. 5,744,161). Piperine is available commercially under the brand name Bioperine® (Sabinsa Corp., Piscataway, N.J.). Natural bioavailability enhancers can be present in an amount of from about 0.02% to about 0.6% by weight based on the total weight of the delivery system.

Synthetic bioavailability enhancers are typically based on macrogol glycols and glycerides or polyethylene glycol (PEG). Examples of suitable synthetic bioavailability enhancers include, but are not limited to, Gelucire®, Labrafil® and Labrasol®, Lauroglycol®, Pleurol Oleique®, (Gattefossé Corp., Paramus, N.J.) and Capmul® (Abitec Corp., Columbus, Ohio).

Synthetic bioavailability enhancers are generally an option considered only when one or more of the functional ingredients included in the delivery system is a drug. The amount of synthetic bioavailability enhancer that can be included in the delivery systems is typically defined by the ratio of synthetic bioavailability enhancer to drug(s). This ratio can vary between about 1.0:10.0 and 10.0:1.0. In one embodiment of the present invention, the synthetic bioavailability enhancer to drug(s) ratio varies between about 1.0:10.0 and 5.0:1.0. In another embodiment of the present invention, the synthetic bioavailability enhancer to drug(s) ratio varies between about 1.0:10.0 and 3.0:1.0.

Process for Preparing the Delivery System

In accordance with the present invention, the delivery systems remain flowable at temperatures below 100° C. to allow for full dispersion and incorporation of the functional ingredients into the matrix while minimising or preventing degradation of these compounds. Thus, although the actual methodology used to prepare the delivery systems may vary depending on the individual components selected to make up the matrix, the process of preparing the matrix comprises the step of incorporating the functional ingredient(s) into the matrix at temperatures below 100° C. The method further comprises the step of dispersing at least one of the functional ingredients in the solvent component prior to combining with the other components of the matrix. In one embodiment of the present invention, the process of preparing the matrix comprises the step of combining the solvent component comprising one or more functional ingredient(s) with the other components of the matrix at temperatures below about 75° C. In another embodiment, the process of preparing the matrix comprises the step of combining the solvent component comprising one or more functional ingredient(s) with the other components of the matrix at temperatures below about 65° C.

Various standard methods known in the confectionery manufacturing industry can be used to prepare the delivery systems and selection of the appropriate method is considered to be within the ordinary skills of a worker in the art. Batch processes, such as kettle cooking, as well as continuous processes, such as direct stream injection jet cookers and indirect stream tubular heat exchangers, are suitable for preparing the delivery system.

The following description represents a general method of preparing the delivery system in one embodiment of the present invention.

Briefly, the process comprises the following steps: a blend of the hydrocolloid component and the sugar component, and optionally water, is prepared. A ratio of components is selected that will result in a final product with the desired moisture content (i.e. 10%-40%). The hydrocolloid(s) may be pre-hydrated in water or may be hydrated during this blending step. The blend is heated to a temperature of less than 100° C., for example between 60° C. and 80° C., such that all ingredients are incorporated. Alternatively, the sugar component, and optionally water, can be heated to a temperature of less than 100° C. (for example between 60° C. and 80° C.) prior to addition of the dry or pre-hydrated hydrocolloid(s) under shear. The temperature of the mixture is then reduced to between 50° C. and 80° C. The functional ingredient(s) are dispersed or dissolved in solvent at a temperature below 100° C., for example, at or below 70° C. If required, one or more sources of mono- or divalent cations and one or more pH adjusting agents can be added to either, or both, of the above preparations. The two preparations are then combined. Flavourings and colourings may optionally be added after this step.

As an alternative to adding pH adjusting agents as indicated above, the pH of the matrix can be adjusted, as necessary, after combining the two preparations. Suitable methods of adjusting the pH of food products are known in the art and include, for example, the addition of buffers, acids or bases, such as citric acid, sodium citrate, phosphates, sodium hydroxide, potassium hydroxide or a combination thereof.

As indicated above, the final product has a moisture level between 10% and 40%, for example between 15% and 20%, and a water activity of less than 0.9.

In one embodiment of the invention, the process includes the step of heating the blend of hydrocolloid(s) and the sugar component (and optionally water) to a temperature between about 60° C. and about 70° C. In another embodiment, the process includes the step of heating the sugar component, and optionally water, to a temperature between about 60° C. and about 70° C. prior to addition, under shear, of the dry or pre-hydrated hydrocolloid(s).

One skilled in the art will appreciate that the temperature at which the functional ingredient(s) are dispersed or dissolved in the solvent component will be dependent on the temperature stability of the functional ingredient(s). For example, for functional ingredients that are labile at elevated temperatures, then the step of dispersing or dissolving in the solvent component can be conducted at or below 70° C., for example, at or below 50° C., whereas for functional ingredients that are more temperature stable, the temperature for this step may be increased, for example to between about 70° C. and 100° C. While complete dissolution of the functional ingredient(s) is not critical to the present invention, in some instances, increasing the temperature of the dispersion/dissolution step for a temperature-stable functional ingredient may be desirable in order to fully dissolve the functional ingredient(s), for example, to improve the final texture of the delivery system.

In one embodiment of the present invention, the process includes the step of dispersing or dissolving the functional ingredient(s) in the solvent component at a temperature between about 40° C. and about 65° C. In another embodiment, the process includes the step of dispersing or dissolving the functional ingredient(s) in the solvent component at a temperature between about 40° C. and about 60° C. In a further embodiment, the process includes the step of dispersing or dissolving the functional ingredient(s) in the solvent component at a temperature between about 40° C. and about 50° C.

In an alternate embodiment of the present invention, the process includes the step of dispersing or dissolving the functional ingredient(s) in the solvent component at a temperature between about 50° C. and about 90° C. In another embodiment, the process includes the step of dispersing or dissolving the functional ingredient(s) in the solvent component at a temperature between about 50° C. and about 85° C. In a further embodiment, the process includes the step of dispersing or dissolving the functional ingredient(s) in the solvent component at a temperature between about 60° C. and about 85° C.

The above process allows for the product intermediates at each stage in the process to remain in a flowable state and, as such, the final product can be simply poured into moulds and allowed to set. As noted above, this property allows a variety of packaging options to be employed for the final moulding and/or packaging of the delivery systems. Once the matrix has been prepared as described above, therefore, it can then be moulded by pouring into pre-formed moulds, for example, using the standard Mogul process, by injection-filling of pre-formed moulds, vertical or horizontal form fill and seal, Unifill, Sarong, blister pack or rotary moulding.

One skilled in the art will appreciate, however, that, if necessary, the matrix can also be readily adapted to extrusion methods.

In final form, the delivery systems of the present invention are semi-solid, intermediate moisture systems, having some properties clearly identified with those of jellies and some properties that are similar to the jujube variety of confectioneries. The matrix of the delivery systems is thus formulated to be semi-solid at normal room temperature (i.e. at temperatures between about 20° C. and about 30° C.). It will be readily apparent that depending on the particular components selected for use in the preparation of the matrix, the amount of each to be included in the matrix may need to be manipulated within the ranges indicated in order to achieve a semi-solid, intermediate moisture product. One skilled in the art of confectionery design can readily determine which component(s) will need to be adjusted in order to achieve an end-product with these physical properties.

Similarly, it will be readily apparent to one skilled in the art that variations can be made to the described process dependent on the type and the actual amount of each component used (within the given ranges) in order to obtain an end product with the described properties. For example, if the carbohydrate component is a starch, it is known in the art that the gelatinisation temperature of the starch may be affected when certain sugars and sugar alcohols are used. If required, therefore, starch, hydrated hydrocolloid and the sugar component can be heated above 100° C. to allow full gelatinisation of the starch to occur and the desired moisture content to be reached. The temperature of the mixture can then be reduced to between 50° C. and 80° C. prior to addition of the functional ingredient(s) and optionally flavourings and colourings.

As is known in the art, modified celluloses, such as methylcellulose and hydroxypropyl methylcellulose, have unique properties resulting in the ability to delay hydration of these carbohydrates during preparation processes. Thus, when these compounds are used a “delayed hydration technique” may be employed in which the cellulose is first dispersed in the solvent component of the matrix and then mixed with the other components in aqueous solution. The hydration of the cellulose then takes place gradually as the processing is complete and the moulded matrix cools. Delayed hydration and non-aqueous fluid carrier techniques using modified celluloses are standard in the art.

Similarly, the choice of hydrocolloid can affect the set up temperature of the matrix. The use of a combination of gelatine and gellan, such as a gelatine:gellan ratio of between about 20:1 and about 40:1, as the hydrocolloid, for example, results in a matrix set-up temperature of about 35° C., as does a combination of gelatine and pectin at a ratio between about 15:1 and about 25:1. In contrast, the use of other hydrocolloids or combinations of other hydrocolloids with or without gelatine or gellan, alters the set up temperature of the matrix. For example, the use of locust bean gum or carageenan results in set up temperatures of around 60° C. The choice of hydrocolloid is thus dependent on the functional ingredient(s) to be incorporated into the matrix. Temperature sensitive functional ingredients will require a hydrocolloid or hydrocolloid mixture that provides a low set up temperature (such as the gelatine:gellan mixture described above), whereas other hydrocolloids or mixtures thereof can be used with functional ingredients that can tolerate higher temperatures.

The manner in which the individual components are combined may also be varied although typically at least one of the functional ingredients is dispersed in solvent prior to addition to the remainder of the components. For example, the hydrocolloid and part of the sugar component can be mixed and heated prior to being blended with the carbohydrate and remainder of the sugar component. Alternatively, the carbohydrate and the sugar component can be mixed and heated prior to addition of the hydrated hydrocolloid, or the carbohydrate maybe added to the solvent component and then blended with the hydrocolloid and sugar component. Likewise, any additional functional ingredients that are to be included in the delivery system and are not dispersed or dissolved in the solvent component can be dispersed or dissolved in one of the other components of the delivery system. For example, one or more functional ingredients can be dispersed or dissolved in water, with or without heating depending on the solubility and temperature stability of the functional ingredient(s), and then combined with the other components of the matrix. These and other variations are considered to be within the scope of the present invention.

In one embodiment of the present invention, the matrix is prepared using (a) modified starch; (b) gelatine:gellan as the hydrocolloid; (c) a mixture of corn syrup and high fructose corn syrup as the sugar component, (d) a mixture of glycerol and propylene glycol as the solvent component, (e) potassium citrate as a source of monovalent cations, and (f) water. The process comprises blending the glycerol and propylene glycol, adding the functional ingredient(s) and warming the resulting blend to 65-70° C. The fructose syrup is blended with water and warmed to 60° C. The gelatine is blended with the gellan, added to the fructose syrup with constant agitation and the temperature is raised to 75° C. in order to dissolve all the components. The corn syrup is warmed to 30-35° C. and the starch and potassium citrate, and optionally other sweeteners, are blended in. The gelatine:gellan blend and the starch blend are then combined and the solution is maintained at 75-80° C. in order to reduce the moisture content to the desired solids content level. The solids content can be measured using standard techniques, such as measurement of the refractive index to estimate production moisture level. Once the desired level has been achieved, the functional ingredient/solvent blend is added, together with any desired colouring and flavouring. The resulting matrix is then moulded using standard procedures.

In another embodiment of the present invention, a matrix containing the same components as indicated above is prepared by the following process. Glycerol and propylene glycol are blended together, the functional ingredient(s) is added and the resulting solution is blended and warmed to 40° C.-60° C. The corn and fructose syrups are blended with water and heated. The dry ingredients are blended and combined with the warmed syrups. The mixture is then heated to at least 80° C. In an alternative embodiment, the blended dry ingredients are blended in with simultaneous live steam injection to reach at least 80° C. The solid content is then adjusted by addition of water to provide a final moisture content of 10% to 30%. The temperature of the syrup mixture is lowered to between 50° C. and 80° C. and the functional ingredient/solvent blend is incorporated. Finally, colouring and flavouring is added, if desired. The matrix is then injection filled into preformed packaging.

In a further embodiment of the present invention, the matrix is prepared using (a) modified starch; (b) gelatine:pectin as the hydrocolloid; (c) a mixture of maltitol syrup and high fructose corn syrup as the sugar component, (d) a mixture glycerol and propylene glycol as the solvent component, (e) potassium citrate as a source of monovalent cations, and (f) water. The process comprises blending the solvents, adding the functional ingredients and warming the mixture to 60° C.-70° C. The starch, gelatine and pectin are blended together with any additional sweeteners required. This blend is added to the syrups with constant agitation and the temperature is maintained at 60° C.-70° C. until the moisture content reaches the desired level. Colouring or flavouring is then added, if desired, and the resulting matrix is moulded using standard techniques.

Testing the Delivery System

1. Physical Properties

One skilled in the art will appreciate that molecular interaction between one or more of the functional ingredient and the matrix may affect the physical attributes of the final product. As is standard in the art, therefore, a sample of the delivery system incorporating the desired functional ingredient(s) can be prepared prior to large-scale production and tested in order to determine whether the matrix retains the desired physical properties, i.e. that the functional ingredients are substantially uniformly dispersed, that degradation of these compounds during the preparation of the matrix is below 20% and that the water activity of the delivery system is below 0.9.

For example, dispersion of the functional ingredient(s) in each delivery system can be determined by dividing a single unit of the final delivery system into several subunits and analysing the content of functional ingredient(s) in each subunit, for example as a % by weight. The levels of functional ingredients can readily be measured by standard analytical techniques such as mass spectrometry, UV or IR spectrometry, or chromatographic techniques, such as gas chromatography or high-performance liquid chromatography (HPLC). If the % by weight of functional ingredient in each subunit is similar, then the functional ingredient is said to be substantially uniformly dispersed throughout the product. One skilled in the art will appreciate that the % by weight need not be identical for each subunit to indicate substantially uniform dispersion. In accordance with the present invention, the % by weight of functional ingredient for each subunit of the final delivery system varies by less than 2%. In one embodiment, the % by weight of functional ingredient for each subunit of the final delivery system varies by less than 1.5%. In other embodiments, the % by weight of functional ingredient for each subunit varies by less than 1% and by less than 0.5%.

Similarly, the degradation of the functional ingredients can be determined by standard analytical techniques taking into account the total amount of each compound included in the preparation of the matrix. Many functional ingredients degrade to yield specific breakdown products, the presence or absence of which can be determined in the final product. As an example, the functional ingredient creatine is hydrolysed to creatinine, which can be distinguished from creatine using chromatographic techniques, such as HPLC. As indicated above, the degradation of the functional ingredients is minimised during the preparation of the delivery system and is less than about 20% in the final product.

The water activity (a_{w}) of the final product can also be analysed by standard techniques. The a_{w} of a food product is a physical property that has direct implications on the microbial safety of the product and influences storage stability. Lower a_{w} values generally indicate a food product that is more stable and more resistant to microbial contamination than one with a high a_{w} value due to the requirement for water of most microbes and the fact that most deteriorative processes in food products are mediated by water. As is known in the art, the a_{w} value of a food product is the ratio of the water vapour pressure of the product (p) to that of pure water (pₒ) at the same temperature, i.e. a_{w}=p/pₒ. In accordance with the present invention, the water activity of the final delivery system is less than about 0.9, for example between about 0.5 and about 0.7.

Other parameters, such as the release rate of the functional ingredients from a delivery system can also be tested by standard methods (for example, the USP Basket Method or Paddle Method; see U.S. Pharmacopoeia XXII (1990)). Typically, a sample of the delivery system containing a known amount of functional ingredient(s) (for example, a unit dose) is placed in an aqueous solution of a predetermined pH, for example around pH 1.2 to simulate stomach conditions and/or around pH 7.4 to simulate colon conditions. The suspension may or may not be stirred. Samples of the aqueous solution are removed at predetermined time intervals and are assayed for their content of the bioactive by standard analytical techniques, such as those indicated above.

In addition, the delivery system may undergo testing to evaluate such factors as the microbial content of the product and the shelf-life of the product. Such quality control testing is standard in the art and can be conducted using known methods.

For example, microbial analysis of the delivery system can be conducted using techniques approved by the appropriate regulatory board, such as those described in “The Compendium of Analytical Methods: HPB Methods for the Microbiological Analysis of Foods” issued by the Health Products and Food Branch of Health Canada. Shelf life is typically evaluated using accelerated shelf life tests in which the stability of the system and the degradation of the functional ingredients contained therein is analysed under conditions that are known to accelerate the degradation of food products and can be correlated to the stability of the product under normal storage conditions.

Texture measurements can also be made to determine whether the delivery system has the required gel strength/hardness. Gel strength or hardness can be measured either directly (expressed as grams force) and indirectly (expressed as a viscosity), or both.

Methods of measuring gel hardness are known in the art. For example, a Kramer single blade shear cell can be used. In this test, a shear blade is driven down at a constant speed through a sample of the delivery system and the peak force as the blade cuts through the sample is measured. The test force is typically reported in kilograms-force. Various machines are available to conduct such testing, for example, a Universal Testing machine such as that available from Instron or Stable Micro Systems (e.g. the Model TA.HD Texture Analyzer).

Gel hardness can also be measured using a standard Brookfield viscometer (e.g. the Model RVDV), which measures the force required to cut through a gelled liquid. A spindle rotating at a set speed is slowly lowered into a sample of the delivery system and the torque required for the spindle to “cut” through the sample is measured. Temperature is important to obtain an accurate viscosity reading and thus the samples are usually tempered to 21° C. to 24° C. prior to testing. The cutting force or torque reading on the viscometer is an empirical measure of gel strength and is reported in centipoise (cps).

Another method useful for measuring sensory texture utilizes the Hamann Torsion/Vane Gelometer. This system provides fracture shear stress and shear strain values and real time test graphs of stress vs. strain or angular deformation. Stress (strength) and strain (deformability) are not “geometrically coupled” as in most traditional (empirical) textural tests, therefore, the strain measurement remains unaffected by the magnitude of the stress measurement. Strain has been found to be the best indicator of gelling quality for proteins and hydrocolloids, as this parameter is less sensitive to concentration effects, and is also a good indicator of the perceived “rubberiness” of food gels. Strain values also predict machining characteristics of food gels, such as ease of slicing. Furthermore, the sample shape does not change during testing with the Torsion Gelometer, thus minimal fluids will be forced from the sample during testing and the gel itself is tested rather than a dehydrated derivative. The mode of failure in torsion testing yields important information about the texture of the sample. Test samples of the delivery system are formed in either cylindrical moulds (tubes) for subsequent milling, which eliminates surface skin effects, or in a dumbbell mold. Samples are then cut to a standard length (for example, 1 inch) and loaded into the measuring cell for testing. Data collection continues for a time past the breaking of the sample (peak stress or Fracture Point). Stress (in kPa), strain, rigidity modulus (G=stress/strain) and slope ratio at failure can be measured in this method.

Palatability can also be tested using standard techniques. Methods of evaluating the organoleptic properties of foods are well-known in the art. For example, sensory evaluations can be performed using individuals who are spatially separated from each other, for example, in individual partitioned booths, as testers and a hedonic nine-point scale that ranges from 1 (most disliked) to 9 (most liked), with 5 indicating no preference [Larmond, Laboratory methods for Sensory Evaluation of Foods, Research branch of Agriculture Canada (1977)]. Odour and taste are generally evaluated under a red light, which masks any differences in the colour of the product. Another nine-point hedonic scale test can be carried out under normal light to evaluate the acceptability of the appearance of the product.

The final product can also be assessed by methods such as those described above for the acceptability of its texture or “mouthfeel.” In one embodiment of the present invention, the texture of the final delivery system is similar to a piece of soft liquorice or a jujube.

2. Efficacy

The various delivery systems of the present invention also can be optionally tested for efficacy in vivo. Typically, when such testing is conducted, efficacy is assessed by bioavailability studies using standard techniques in the pharmaceutical art, such as peak plasma levels and pharmokinetic analyses (see, for example, Enna, et al., Current Protocols in Pharmacology, J. Wiley & Sons, New York, N.Y.).

Bioavailability studies are usually conducted by administering to groups of subjects various doses of the delivery system under study over a pre-determined period of time and comparing plasma levels of the functional ingredients in these groups at varying intervals with an appropriate control or controls. Appropriate controls include groups of subjects taking recommended doses of competitor's products. The subjects may or may not have fasted prior to administration of the doses of the delivery system. Single dose or multiple dose studies may be conducted. The studies can also be used to monitor any side-effects of the dosing regimens of the delivery system under investigation by compiling reports of any adverse effects encountered during the course of the study and comparing them to side-effects reported by the control group(s). Optionally, optimal dosing schedules can also be determined in this manner.

Studies to determine that the combination of functional ingredients in a delivery system bring about the desired effect in a subject can also be conducted in a similar manner to the bioavailability studies indicated above. Such studies are routine in the art and can be readily designed and conducted by a skilled technician. End effects are measured dependent on the type of effect the delivery system is intended to bring about. For example, for weight loss or thermogenic delivery systems, the body weight and/or body fat percentage of individual subjects to whom varying doses of the delivery system is being administered can be monitored over a period of time and compared to that of individuals in control groups, for example, placebo groups or groups taking competitor's products. For muscle enhancement delivery systems, criteria such as percentage increase in muscle mass can be monitored, for bone health formulations, criteria such as bone density can be monitored. Other factors and end effects that can be monitored for various formulations will be readily apparent to one skilled in the art.

In addition, for certain specific functional ingredients, characteristic metabolic products can be analysed. For example, the effect of creatine on muscle phospho-creatine can be measured by performing muscle biopsy on individuals following a controlled dosing regimen. Extraction and measurement of phosphorus compounds from the biopsy using standard techniques is then conducted to determine changes in muscle phosphor-creatine. Non-invasive measurements, for example, using ³¹P-NMR to measure changes in phosphorus compounds can also be utilized. The total concentration of creatinine can also be measured after 24 hours in order to examine clearance of creatine.

Format of the Delivery System

The present invention contemplates various formats for the delivery systems. For example, the delivery systems may be in the form of a confectionery, such as a jujube, in which case it may be formulated alone or it may further comprise a coating, such as a chocolate or yoghurt coating. Preparation of jujube or jelly type confectionery products are known in the art and include, for example, the use of moulds, injection-filling of pre-formed packages and extrusion processes. It will be readily apparent to one skilled in the art that such standard techniques can be applied to prepare a wide variety of different shaped confectioneries.

Methods of making and applying coatings to confectionery products are also well-known in the art. Coatings are in general compound coatings the major ingredients of which are sugar and fat. Flavours and colours are often added. Chocolate coatings are usually based on cocoa butter whereas yoghurt coatings typically comprise powdered yoghurt. In general, the coating material comprises a fat that is solid at room temperature, but liquid at temperatures in excess of, for example, 35° C., together with other materials that confer appropriate organoleptic attributes on the final coating. Typically, application of the coating to the confection takes place while the coating is molten, for example, by passing the formed confection simultaneously through a falling curtain of liquid coating and over a plate or rollers which permit coating to be applied to the under surface of the confection. Excess coating is blown off by means of air jets and the coated confection passes through a cooling tunnel where refrigerated air currents solidify the applied coating. In accordance with the present invention, the properties and method of application of the coating must not interfere with, or compromise, the properties of the delivery system. For example, the application of the coating must not require elevated temperatures that would affect the stability of the functional ingredient(s) incorporated into the delivery system.

The present invention further contemplates the delivery system as a filling or a coating, for example, for baked goods such as wafers or cookies. For example, the matrix can be used as a layer between two wafers, or a jelly layer on the top of a cookie or sponge, in which case the product may be further coated with a chocolate or other flavoured coating, if desired, as described above for confectionery products. Alternatively, the matrix may be used to fill doughnut type baked goods. Methods of filling and coating baked goods are also well known in the art.

Administration and Use

As described above, the delivery systems of the present invention can be formulated to accommodate specific combinations of functional ingredients in order to produce or elicit specific physiological effects. For example, drug delivery systems can be formulated to contain certain combinations of therapeutic or diagnostic agents, or combinations of nutritional supplements. A wide variety of other combinations of functional ingredients are known in the art for providing specific physiological benefits and are suitable for inclusion in a delivery system of the invention. Non-limiting examples are provided in Table 1. Other exemplary delivery systems contemplated by the present invention include, but are not limited to, delivery systems formulated with combinations of functional ingredients to promote sexual potency, promote endurance, promote cardiovascular health, control fat and/or cholesterol, promote healthy joints, maintain or improve bone density, enhance cellular anti-oxidant capacity, control appetite, to promote energy, increase endurance, promote weight loss, promote muscle enhancement, improve digestion, help prevent colds, fight infection, produce thermogenic effects, or enhance memory. For example, combinations of ephedra alkaloids and caffeine are known in the art to produce a thermogenic effect and can be included in a thermogenic delivery system. Similarly combinations of Ginkgo biloba and Goto kola are used for memory enhancement and can be included in a memory enhancement delivery system.

As will be readily apparent to one skilled in the art, many of the exemplary categories outlined above overlap and are not mutually exclusive. Thus, delivery systems can be designed in accordance with the present invention that can bring about more than one desired physiological effect.

The selected functional ingredients are incorporated into the delivery system at levels sufficient to affect the structure or function of the body when taken regularly. Such levels are known in the art or can readily be determined by a skilled technician. It is understood that the total daily intake may be based on administration of one unit of the delivery system, or it may be based on administration of more than one unit. The amount of functional ingredients in the final product will thus vary depending on the format of the units and the number to be administered daily.

The delivery systems of the invention can be formulated in various unit sizes depending on the amount of functional ingredient(s) to be incorporated therein and on requirements of the target consumer. The delivery systems of the present invention can be formulated to have a unit size between about 2 grams and about 30 grams, for example between about 3 grams and about 30 grams. In one embodiment, a unit of the delivery system is between about 3 grams and about 20 grams. In another embodiment, a unit of the delivery system is between about 3 grams and about 15 grams. In another embodiment, a unit of the delivery system is between about 3 grams and about 10 grams. Where appropriate, the delivery systems can be provided in a multi-dose format that is pre-scored into unit doses.

The organoleptic properties of the delivery systems of the present invention ensure that they are easy to take and/or to administer. In one embodiment, the delivery systems are formulated for administration to humans and thus contain flavours that would appeal to humans, such as fruit-based flavours. Delivery systems of the present invention that are formulated with confectionery-like qualities and flavours are also appealing to children who are often resistant to taking medications or supplements due to unpleasant tastes or mouthfeel. Thus, in another embodiment, the delivery systems provide a means of easily administrating certain functional ingredients, such as multi-vitamins and minerals, to children.

In another embodiment, the delivery systems are formulated for administration to a non-human animal. In a related embodiment the non-human animal is a domestic animal, such as a dog or a cat. Administration of functional ingredients to an animal in conventional solid dosage forms, such as tablets and capsules, can be problematic in that the animal often expels them, and multiple dosing is often difficult because the animal learns to resist the dosing procedure. It will be readily apparent that the delivery systems of the present invention, which is formulated as a foodstuff, is ideally suited for administration of functional ingredients to animals. When formulated for this purpose, the matrix may contain flavours that more typically appeal to non-human animals, for example, fish or meat flavours. Additional functional ingredients more suited to animal use, such as dessicated liver, may also be included.

Kits

The present invention additionally provides for kits containing a delivery system for administration to a human or non-human animal. The kits would provide an appropriate dosing regimen for a prescribed period for the functional ingredients contained in the delivery system.

The kits of the invention comprise one or more packages containing the delivery system optionally in combination with a set of instructions, generally written instructions, relating to the use and/or dosage of the functional ingredients contained in the delivery system. The instructions can include information as to the appropriate dosage and dosing schedule for the functional ingredients in terms of units of the delivery system. The packages containing the delivery system may in the form of unit doses, bulk packages (for example, multi-dose packages) or sub-unit doses. The doses may be packaged in a format such that each dose is associated, for example, with a day of the week. There may also be associated with the kit a notice in the form prescribed by a governmental agency regulating the manufacture, use or sale of biological products, which notice reflects approval by the agency of manufacture, use or sale for human or animal administration.

To gain a better understanding of the invention described herein, the following examples are set forth. It will be understood that these examples are intended to describe illustrative embodiments of the invention and are not intended to limit the scope of the invention in any way. All percentages throughout the specification and claims are by weight of the final delivery system unless otherwise indicated.

### EXAMPLES

### Example 1

### Delivery System for Creatine and Dimethylglycine

One example of a delivery system containing creatine and dimethylglycine is as follows:

| Ingredient | % by Weight |
|---|---|
| Glycerol | 14.57% |
| Propylene Glycol | 5.30% |
| Creatine monohydrate | 11.71% |
| Corn Syrup 62DE | 31.79% |
| Sucralose | 0.04% |
| Modified Starch (Staley Softset ®) | 2.65% |
| Potassium citrate | 2.15% |
| Dimethylglycine | 1.67% |
| High fructose corn syrup | 9.27% |
| Water | 14.57% |
| Gelatine 100 bloom type B | 1.32% |
| Gelatine 250 bloom type A | 3.97% |
| Gellan (Kelcogel ® LT100) CP Kelco | 0.32% |
| Colour | 0.21% |
| Flavour | 0.45% |
| Total: | 100.00% |

Glycerol and propylene glycol were first blended and the creatine was added. The blend was heated to 65-70° C. In a separate container, the two types of gelatine and the gellan were blended together. The fructose syrup and water were mixed and heated to 60° C., after which the gelatine:gellan mixture was added with constant agitation. The mixture was then heated to 75° C. to allow the components to dissolve. In a third container, the corn syrup was warmed to 30-35° C. and the sucralose, potassium citrate, dimethylglycine and starch were then blended in. The corn syrup mixture was combined with the gelatine:gellan mixture and heated to 75-80° C. until the moisture content was reduced and the desired solids level achieved. The creatine mixture was then added together with the colour and flavour additives. The delivery system was then moulded using standard techniques.

### Example 2

### Heart Health Delivery System

One example of a delivery system for a formulation to promote heart health is as follows:

| Ingredient | % by Weight |
|---|---|
| Glycerol | 12.57% |
| Propylene Glycol | 4.19% |
| Arginine | 14.02% |
| Maltitol solution | 33.52% |
| Modified Starch (Staley Miraquick ®) | 2.79% |
| Potassium citrate | 1.17% |
| Sucralose | 0.04% |
| High fructose corn syrup | 9.78% |
| Water | 15.37% |
| Gelatine 250 bloom type A | 5.59% |
| Gellan (Kelcogel ® LT100) CP Kelco | 0.28% |
| Colour | 0.168% |
| Flavour | 0.503% |
| Total: | 100.00% |

Glycerol and propylene glycol were first blended and the arginine was added. The blend was heated to 65-70° C. In a separate container, the gelatine and the gellan were blended together. The fructose syrup and water were mixed and heated to 60° C., after which the gelatine:gellan mixture was added with constant agitation. The mixture was then heated to 75° C. to allow the components to dissolve. In a third container, the maltitol solution was warmed to 30-35° C. and the sucralose, potassium citrate and starch were then blended in. The maltitol mixture was combined with the gelatine:gellan mixture and heated to 75-80° C. until the moisture content was reduced and the desired solids level achieved. The arginine mixture was then added together with the colour and flavour additives. The delivery system was then moulded using standard techniques.

### Example 3

### Energy Delivery System

An example of a delivery system containing a formulation of functional ingredients to promote energy is as follows:

| Ingredient | % by Weight |
|---|---|
| Glycerol | 13.82% |
| Propylene Glycol | 5.53% |
| Creatine monohydrate(CM) | 4.59% |
| Conjugated Linoleic Acid (CLA) | 4.59% |
| Lecithin | 1.05% |
| Isomalt syrup | 33.17% |
| Sucralose | 0.055% |
| Modified Starch (Staley Softset ®) | 2.76% |
| Potassium citrate | 2.24% |
| N,N, dimethylglycine (dmg) | 0.47% |
| Rhodiola/Seabuckthorn extract | 0.21% |
| solution | |
| Chromium chelate | 0.11% |
| High Fructose Corn syrup | 9.68% |
| Water | 15.20% |
| Gelatine 250 bloom type A | 5.53% |
| Gellan (Kelcogel ® LT100) CP | 0.33% |
| Kelco | |
| Colour | 0.08% |
| Flavour | 0.08% |
| Total: | 100.00% |

The CLA, creatine and lecithin were first mixed together. The glycerol and propylene glycol were mixed and heated to 65-70° C. The CLA/creatine/lecithin blend was then added to the solvents and the resultant mixture was maintained at 65-70° C. In another container, the gelatine was mixed with the gellan. The fructose syrup and water were combined and heated to 60° C. and the gelatine:gellan mixture was then added, after which the temperature was raised to 75° C. and maintained at this temperature until the solids dissolved. In another container, the isomalt syrup was warmed to 30-35° C. and the sucralose, citrate, dmg, rhodiola/seabuckthorn extract, chromium chelate and starch were then blended in. This mixture was combined with the gelatine mixture and the temperature maintained at 75-80° C. until the moisture content was reduced sufficiently to give the desired solids level. Once the proper moisture level was achieved, the glycerol-glycol mixture was blended in together with colour and flavouring additives. The mixture was then moulded using standard techniques.

### Example 4

### Delivery System for Creatine

One example of a delivery system for creatine is as follows:

| Ingredient | % by Weight |
|---|---|
| Glycerol | 27.9990% |
| Propylene Glycol | 3.4145% |
| Potassium Hydroxide | 0.1208% |
| Creatine Monohydrate | 24.0154% |
| High Fructose Corn Syrup | 15.7068% |
| Corn syrup | 14.7962% |
| Starch (Mira-quik MGL ™) | 2.5040% |
| Water | 3.9836% |
| Potassium phosphate | 0.4234% |
| Sucralose | 0.0381% |
| Potassium citrate | 0.9526% |
| Gelatine Type A | 4.7803% |
| Pectin | 0.2732% |
| Flavour | 0.5464% |
| Colour | 0.2982% |
| Total: | 100.0000% |

Glycerol and propylene glycol were first blended and the creatine was added. The blend was heated to 45-50° C. In a separate container, the gelatine, pectin, starch and sucralose were blended together. The fructose and glucose syrups and water were mixed and heated to 60° C., after which the salts and pH modifying agents were added with constant agitation and heated to 60-70° C. to dissolve the solids. The powder blend was then incorporated into the syrup mixture using high shear. Finally, the creatine mixture was added, together with the colour and flavour additives, and blended. The delivery system was then moulded using standard techniques.

### Example 5

### Weight Loss or Maintenance Delivery System

An example of a delivery system containing a combination of functional ingredients to aid in weight loss or maintenance is as follows:

| Ingredient | % by Weight |
|---|---|
| Glycerol | 16.67% |
| Propylene Glycol | 7.86% |
| Conjugated linoleic acid - Clarinol 80 | 7.86% |
| Citrus Aurantium | 0.50% |
| Maltitol syrup | 35.86% |
| High fructose corn syrup | 15.73% |
| Sucralose | 0.06% |
| Modified Starch (Staley Miraquick ®) | 3.15% |
| Potassium citrate | 1.42% |
| Potassium hydroxide | 0.92% |
| Inulin | 0.63% |
| Caffeine | 0.25% |
| Mixed tocopherols | 0.04% |
| Ascorbic acid | 0.03% |
| Water | 1.38% |
| Gelatine | 6.29% |
| Pectin | 0.31% |
| Colour | 0.3% |
| Flavour | 0.74% |
| Total: | 100.00% |

The glycerol and propylene glycol were first blended together. The CLA, Citrus aurantium and mixed tocopherols were then added and the resultant mixture was warmed to 60-70° C. In another container, the syrups, water, potassium citrate and potassium hydroxide were combined and warmed to 60-70° C. The starch, gelatine, pectin, inulin and sucralose were pre-blended then added to the syrup mixture under high shear. This mixture was combined with the glycerol mixture and the temperature maintained at 60-70° C. until the moisture content was reduced sufficiently to give the desired solids level. Colour and flavour were added and the mixture was then moulded using standard techniques.

### Example 6

### Delivery System for Creatine

Another example of a delivery system containing creatine is as follows:

| Ingredient | % by Weight |
|---|---|
| Glycerol | 14.82% |
| Propylene Glycol | 5.39% |
| Creatine monohydrate | 11.91% |
| Corn Syrup 62DE | 32.33% |
| Sucralose | 0.04% |
| Modified Starch (Staley Softset ®) | 2.70% |
| Potassium citrate | 2.19% |
| High fructose corn syrup | 9.43% |
| Water | 14.82% |
| Gelatine 100 bloom type B | 1.34% |
| Gelatine 250 bloom type A | 4.04% |
| Gellan (Kelcogel ® LT100) CP Kelco | 0.33% |
| Colour | 0.21% |
| Flavour | 0.46% |
| Total: | 100.00% |

Glycerol and propylene glycol were first blended and the creatine was then added. The blend was heated to 65-70° C. In a separate container, the two types of gelatine and the gellan were blended together. The fructose syrup and water were mixed and heated to 60° C., after which the gelatine:gellan mixture was added. The mixture was then heated to 75° C. to allow the components to dissolve. In a third container, the corn syrup was warmed to 30-35° C. and the sucralose, potassium citrate, and starch were then blended in. The corn syrup mixture was combined with the gelatine:gellan mixture and heated to 75-80° C. until the moisture content was reduced and the desired solids level achieved. The creatine mixture is then added together with the colour and flavour additives. The delivery system is then moulded using standard techniques.

### Example 7

### HPLC Analysis of Creatine Stability

Samples of the delivery system produced by the method described in Example 6 were analyzed by high performance liquid chromatography (HPLC) using UV detection to determine the percentage of creatine. Prior to injection, each sample was subject to a dissolution procedure wherein the sample was cut into small pieces and heated in 400 ml of Type 1 water at 90° C. for 10 minutes. The samples were then transferred to a water bath at 4° C. and 50 ml of 1% perchloric acid was added. The mixture was then heated to 28° C., transferred to a 500 ml volumetric flask and the volume made up to 500 ml with Type 1 water. A 60 μL aliquot of this solution was then added to 140 μL of methanol and vortexed. Three replicates were prepared for each sample. Samples of 10 μL of the final solution were used to inject into the HPLC.

The percentage of creatine (by weight) was determined by comparing the mean response of creatine in each sample to the mean response of a stock solution at known concentrations. For each replicate prepared as described above, the solution was injected in triplicate.

Tables 3 and 4 outline the quantity and percentage creatine in the samples of the delivery system. Of particular note is the only slight variation between the percentage creatine by weight of each jujube despite the larger variation in the weight of the jujubes. The percentage by weight of creatine determined for each jujube varied between 7.71% and 9.04% (% CV=14.1%), while the weight of the jujubes varied from 7082.40 mg to 11124.16 mg. The mean percentage creatine by weight for the samples was 8.0%. This is consistent with the expected amount of 9% of chelate in the final product.

TABLE 2
Peak Height Responses and Determined Quantity (Mg) of Creatine Monohydrate Chelate
in Jujubes

| | Reference | Jujube No. | | | | | | | | |
|---|---|---|---|---|---|---|---|---|---|---|
| | Stock | 1 | 2 | 3 | 4 | 5 | 6 | 7 | 8 | 9 |
| | 394.09 | 452.48 | 570.96 | 589.83 | 622.90 | 600.57 | 477.41 | 618.16 | 530.70 | 648.05 |
| | 388.77 | 481.39 | 563.36 | 602.88 | 635.36 | 631.99 | 488.51 | 628.59 | 537.26 | 649.14 |
| | 385.00 | 505.71 | 601.46 | 598.41 | 636.37 | 648.53 | 457.92 | 615.64 | 527.72 | 630.77 |
| MEAN | 389.29 | 479.86 | 578.59 | 597.04 | 631.54 | 627.03 | 474.61 | 620.80 | 531.89 | 642.65 |
| S.D. | 4.57 | 26.65 | 20.16 | 6.63 | 7.50 | 24.36 | 15.49 | 6.87 | 4.88 | 10.31 |
| % CV | 1.2 | 5.6 | 3.5 | 1.1 | 1.2 | 3.9 | 3.3 | 1.1 | 0.9 | 1.6 |

| | | | | | | | | | |
|---|---|---|---|---|---|---|---|---|---|
| Creatine | 640.37 | 772.13 | 796.75 | 842.79 | 836.77 | 633.37 | 828.45 | 709.81 | 857.62 |
| Monohydrate Chelate | | | | | | | | | |
| per Jujube (mg)¹ | | | | | | | | | |

¹Calculated as the (Mean Peak Height of Jujube Solutions)/(Mean Peak Height of Reference Stock Solutions) × (1039 ug/mL) × (500 mL)/(1000)

TABLE 4
Percentage Creatine Monohydrate Chelate by Weight in Jujubes

| | | Determined | |
|---|---|---|---|
| | | Concentration | % Creatine |
| | | of Creatine | Monohydrate |
| | | Monohydrate | Chelate |
| Jujube No. | Weight (mg) | Chelate (mg) | by Weight (%) |
| 1 | 7082.40 | 640.37 | 9.04 |
| 2 | 9620.96 | 772.13 | 8.03 |
| 3 | 10299.80 | 796.75 | 7.74 |
| 4 | 10583.38 | 842.79 | 7.96 |
| 5 | 10535.61 | 836.77 | 7.94 |
| 6 | 7895.14 | 633.37 | 8.02 |
| 7 | 10434.55 | 828.45 | 7.94 |
| 8 | 9095.45 | 709.81 | 7.80 |
| 9 | 11124.16 | 857.62 | 7.71 |
| MEAN | 9630.16 | 768.67 | 8.02 |
| S.D. | 1362.14 | 87.07 | 0.40 |
| % CV | 14.1 | 11.3 | 5.0 |

### Example 8

### In vivo Testing I

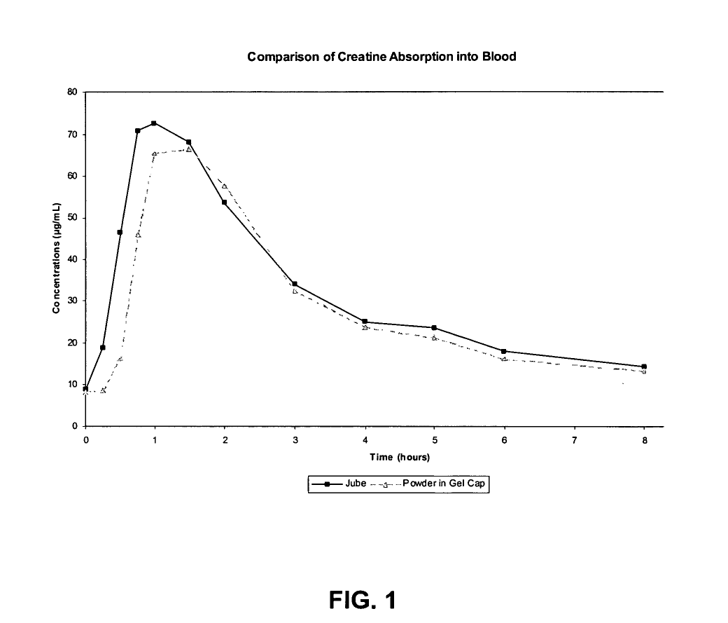

Serum concentration levels of creatine of subjects who ingested either 3.5 gram of micronized creatine powder in capsule format or 3.5 gram of micronized creatine in jujubes (prepared as described in Example 4) were analysed by mass spectroscopy. Seven individuals were enrolled in the test, with an age range between 18 and 50 years. Individuals fasted overnight prior to administration of the creatine. The test protocol was as follows. Individuals were administered jujube containing 3.5 g creatine with 8 oz water. Blood samples were taken every 15 minutes for the first hour, every 30 minutes for the second hour and subsequently at hourly intervals for a total of 8 hours after administration. After sufficient period of time to allow blood creatine levels to return to normal, the subjects were administered 5 capsules containing a total of 3.5 g creatine with 8 oz water. Blood samples were taken at the same time intervals as indicated above. Results are shown in FIG. 1.

### Example 9

### In vivo Testing II

Human serum concentration levels of creatine in subjects who ingested jujubes prepared as described in Example 6 were analysed by HPLC using mass spectroscopy (MS) detection.

In one study, during a period of four days, serum samples from one subject who consumed either (1) 1 gm of creatine monohydrate in a jujube (Day 1A); (2) 500 mg creatine monohydrate/500 mg creatine chelate in the form of a ‘mixed’ jujube (Day 1B); (3) 1 gm creatine monohydrate powered drink (Day 2A); or (4) 500 mg creatine monohydrate/500 mg creatine chelate powered drink (Day 2B). For the entire study, serum samples were taken over a period of six sampling times. The subject fasted for eight hours prior to dosing.

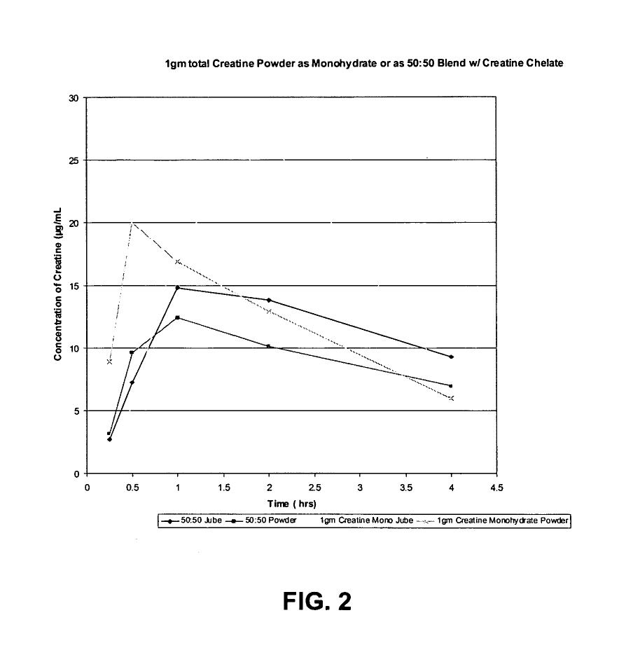

Samples were stored at −20° C.±10° C. for the duration of the analysis. The serum samples were prepared by first adding 50 uL of an internal standard and 20 uL of a 50% perchloric acid solution to 250 uL of the sample, after which they were centrifuged. The supernatant of each sample was then injected into the HPLC/MS system for analysis. The results are plotted in FIG. 2.

The results show that higher serum levels of creatine concentrates were achieved when the subject consumed 1 gm of the creatine monohydrate contained in the jujube compared to values obtained when the subject consumed the creatine powered drinks or the ‘mixed’ jujube containing both creatine monohydrate and creatine chelate. Additionally, serum creatine levels were also capable of being maintained for a longer period of time when the subject consumed the jujube containing creatine monohydrate. The higher serum creatine level over a longer period of time was also noted as creatine levels were still elevated after two hours following ingestion of the creatine monohydrate jujube.

### Example 10

### Delivery System for Creatine

Another example of a delivery system containing creatine is as follows:

| Ingredient | % by Weight |
|---|---|
| Glycerol | 15.97% |
| Propylene Glycol | 5.51% |
| Creatine Monohydrate | 16.71% |
| 63 DE Corn syrup | 21.20% |
| High Fructose Corn Syrup | 24.78% |
| Gelatine 250 Bloom Type A | 5.51% |
| Gellan | 0.33% |
| Sucralose | 0.06% |
| potassium citrate | 1.40% |
| Modified Starch (Staley Miraquick ®) | 2.75% |
| Water | 4.96% |
| Flavour | 0.56% |
| Colour | 0.28% |
| Total: | 100.00% |

Creatine was added to a mixture of glycerol and propylene glycol, and heated to 40-60° C. The syrups were blended with water and the dry ingredients were mixed into the syrup mixture. The combined mixture was then heated to at least 80° C. Alternatively, the blended dry ingredients can be blended in with simultaneous live steam injection to reach at least 80° C. The solid content was then adjusted by addition of water if necessary to provide a final moisture content of between about 10% to about 30%. At this point, the temperature of the syrup mixture was lowered to between 50° C. and 80° C. and the glycerol-glycol mixture was added. Colour and/or flavouring additives were then added and the delivery system was injection filled into the preformed packaging.

### Example 11

### HPLC Analysis of Creatine Stability

Samples of the delivery system produced by the method described in Example 10 were analyzed by HPLC using UV detection to determine the percentage of creatine monohydrate by weight of each sample. Prior to injection, each sample was subject to a dissolution procedure wherein the sample was cut into small pieces and heated in 200 ml of water at 90° C. for 10 minutes, then transferred to a water bath at 4° C. The mixture was subsequently heated to 28° C., transferred to a 250 ml volumetric flask and the volume made up to 250 ml with water. After mixing, a 1 ml aliquot of the mixture was placed into an Eppendorf tube and centrifuged at 10 000 rpm. The supernatant was filtered through a 0.2μ filter and centrifuged again at 10 000 rpm. A 5 μl sample of the supernatant was then taken for HPLC analysis. Three injections were made for each sample preparation.

The results of the HPLC analysis are given in Tables 5 and 6. Both the weight of the jujubes and the percentage by weight of creatine contained within each sample are notably uniform. The weight of the jujubes varied from 26262.37 mg to 26954.56 mg, with an average value of 26774.37 mg, and the percentage by weight of creatine varied from 11.75% to 11.85%, with an average value of 11.80%.

TABLE 5
Percentage Creatine Monohydrate by weight in Jujubes

| | | Determined | |
|---|---|---|---|
| | Weight/ | Conc. of Creatine/ | % Creatine |
| Jujubes | mg | mg | by weight |
| 1 | 26954.56 | 3175.55 | 11.78% |
| 2 | 26262.37 | 3110.82 | 11.85% |
| 3 | 25807.23 | 3151.85 | 11.75% |
| 4 | 28925.42 | 3181.04 | 11.81% |
| 5 | 26848.04 | 3168.55 | 11.80% |
| 6 | 26847.58 | 3165.65 | 11.80% |
| Average | 26774.37 | 3159.41 | 11.80% |

TABLE 6
Peak Height Responses of Creatine Monohydrate in Jujubes

| | Peak Area | | | | | |
|---|---|---|---|---|---|---|
| | No. 22 | No. 23 | No. 24 | No. 25 | No. 15 | No. 27 |
| | Jujube 1 | Jujube 2 | Jujube 3 | Jujube 4 | Jujube 5 | Jujube 6 |
| | 25051.20 | 24550.57 | 24829.29 | 25080.93 | 25031.10 | 25010.23 |
| | 25977.39 | 24559.88 | 24921.40 | 25137.22 | 25023.13 | 25027.83 |
| | 25105.90 | 24591.11 | 24922.88 | 25147.54 | 25014.97 | 25024.65 |
| Average | 25078.50 | 24567.18 | 24897.19 | 25121.76 | 25023.07 | 25023.94 |
| Std. Dev | 27.87 | 21.24 | 53.02 | 35.71 | 8.07 | 4.39 |
| CV | 0.1% | 0.1% | 0.2% | 0.1% | 0.0% | 0.0% |

### Example 12

### Accelerated Shelf-Life Determination

An accelerated shelf life test was conducted on the creatine delivery system prepared by the method described in Example 10. Microbial analysis was conducted using approved methods as described in The Compendium of Analytical Methods: HPB Methods for the Microbiological Analysis of Foods (Volume 2) issued by the Health Products and Food Branch of Health Canada. After subjecting samples of the delivery system to a temperature of 35° C. and a relative humidity of 45-55% for a period of 35 days, the samples were tested for the presence of various microorganisms as listed in Table 7. The average water activity of the samples tested was approximately 0.51.

TABLE 7
Microbial Analysis of Creatine Monohydrate Jujubes -
Accelerated Shelf Life Determination

| | HPB Reference | Results (No. |
|---|---|---|
| Test Conducted | Number | Colonies/Gm Product) |
| Total aerobic plate count | MFHPB - 18 | <10 |
| Total coliforms | MFHPB - 34 | <10 |
| E. coli | MFHPB - 34 | <10 |
| Yeast | MFHPB - 22 | <50 |
| Mould | MFHPB - 22 | <50 |
| Yeast Osmophilic | MFHPB - 22 | <50 |
| Mould Osmophilic | MFHPB - 22 | <50 |
| Staphylococcus aureus | MFHPB - 21 | <25 |
| Salmonella | MFHPB - 20 | not detected |

In addition to the above microbial analysis, the creatine level in each sample was determined by HPLC prior to the test and after 35 days. The average creatine content for four samples randomly selected for analysis after 35 days was compared to the average creatine content for three samples taken prior to the shelf life test. HPLC analysis of creatine monohydrate levels was conducted as described in Example 11.

The results, as shown in Table 7, indicate that after a period of 35 days at the above-described conditions, microbial contamination was minimal and well below accepted levels. Based on these results, the delivery system is shown to have a stable shelf life of at least one year from the date of manufacture.

Results from the HPLC analysis also indicated that levels of creatine monohydrate remained stable in the jujubes after 35 days exposure to the above-described conditions. Prior to the start of the experiment, three jujubes had an average of 13.4% by weight of creatine monohydrate. After 35 days, four jujubes were shown to have an average of 14.2% by weight of creatine monohydrate, which is within the error limits of the analysis performed.

### Example 13

### Analysis of Water Activity of the Delivery System

Water activity was measured in samples of jujubes that had been prepared according to the method described in Example 10.

The procedure for measuring water activity is based on the fact that the water activity of a sample is equal to the relative humidity created by the sample in a closed environment when in equilibrium. The procedure uses a water activity meter constructed by David Brookman & Associates (DB&A). The DB&A Water Activity Meter uses an Omega Engineering HX92C Relative Humidity indicator to measure the relative humidity within a closed environment containing the sample. The Omega probe converts the relative humidity (R.H.) into milliamperes (ma), where 4 ma equals 0% R.H. and 20 ma equals 100% R.H. The water activity meter is calibrated to 11.3% R.H. using a saturated solution of LiCl and to 75.3% R.H. using a saturated solution of NaCl.

The samples are manually macerated in a plastic bag and then transferred to a 30 ml sample bottle. The bottles are filled with sample to at least 1 cm from the shoulder. The bottles are capped until use and stored at room temperature. Measurements are taken by screwing the sample bottle onto the DB&A meter probe and the bottle probe assembly is maintained in a vertical position in a rack. Measurements are taken at hourly intervals at room temperature (20-22° C.) until such time that successive readings do not vary more than 1%.

Random sampling of the jujubes was conducted. The water activity (a_{w}) was determined to be 0.507, 0.515 and 0.544. These values are well below levels those that favour the growth of microorganisms. It has been shown that microorganisms generally grow best between a_{w} values of 0.995-0.980 and most microbes will cease to grow at a_{w} values less than 0.900.

### Example 14

### Delivery System for Calcium and Vitamin D

The following delivery system was formulated to deliver about 1.75 g calcium lactate and 3.5 μg Vitamin D in a 5.5 g product. The resulting product comprises over 35% w/w of functional ingredients and less than 14% w/w of sugar syrup. The final moisture content of the delivery system was 16.1%.

| Ingredient | % by Weight |
|---|---|
| Glycerol | 31.6658% |
| Propylene Glycol | 0.9223% |
| Vitamin D (10 0000 IU/g) | 0.0252% |
| Calcium lactate | 35.0476% |
| High Fructose Corn Syrup | 13.8346% |
| Gelatine | 3.8429% |
| Pectin | 0.1783% |
| Sweetening agents | 0.0307% |
| Modified starch | 1.2297% |
| Flavour | 0.1045% |
| Colour | 0.2060% |
| Water | 12.9123% |
| Total: | 100.0000% |

The glycerol and propylene glycol were blended and the calcium lactate and vitamin D dispersed therein and the blend warmed to 40-50° C. The sugar syrups were blended with the water and warmed to 60-70° C. The gelatine, pectin, sweetening agents and other dry ingredients were preblended and introduced into the syrup under shear. The calcium lactate/solvent blend was then uniformly blended with the gelatine preparation. Flavour and colour were added and the whole maintained between 40° C. and 55° C.

### Example 15

### Delivery System for Indomethacin

The following drug delivery system was formulated to deliver 25 mg Indomethacin in a 6 g final product. The delivery system comprises over 38% w/w of solvent (glycerol+propylene glycol).

| Ingredient | % by Weight |
|---|---|
| Glycerol | 35.96% |
| Propylene glycol | 2.11% |
| Indomethacin | 0.42% |
| 63 DE Corn syrup | 19.29% |
| High Fructose Corn Syrup | 22.67% |
| Gelatine | 8.64% |
| Pectin | 0.31% |
| Sweetening agents | 0.12% |
| KOH | 0.42% |
| Modified Starch | 1.94% |
| Flavour | 0.18% |
| Colour | 0.36% |
| Water | 7.59% |
| Total: | 100.00% |

The glycerol and propylene glycol were blended and the indomethacin dispersed therein and the blend warmed to 75° C. The sugar syrups were blended with the water and warmed to 60-70° C. The gelatine, pectin, sweetening agents and other dry ingredients were preblended and introduced into the syrups under shear. The indomethacin/solvent blend was then uniformly blended with the gelatine preparation. Flavour and colour were then added and the whole maintained between 40° C. and 55° C.

Delivery systems including indomethacin in amounts ranging from 25 mg to 200 mg in a 6 g to 13 g final product were made following the protocol described above. The final moisture content of these products ranged from 15.0 to 17.2%, the pH ranged from 5.40 to 6.25, and the a_{w} ranged from 0.52 to 0.59.

### Example 16

### Delivery System for Ibuprofen with a Bioavailability Enhancer

The following drug delivery system was formulated to deliver about 100 mg Ibuprofen in a 3 g final product. The product also contained 3.33% w/w of a bioavailability enhancer (Gelucire). The delivery system was prepared by the method outlined above for Example 15. The final moisture content of the product was between 15.2% and 16.9%, the pH was between 4.93 and 6.20 and the a_{w} between 0.45 and 0.59.

| Ingredient | % by Weight |
|---|---|
| Glycerol | 30.19% |
| Propylene glycol | 2.09% |
| Ibuprofen | 3.33% |
| Gelucire 44/14 | 3.33% |
| 63 DE Corn syrup | 19.24% |
| High Fructose Corn Syrup | 22.56% |
| Gelatine | 8.58% |
| Pectin | 0.31% |
| KOH | 0.26% |
| Sweetening agents | 0.12% |
| Modified Starch | 1.92% |
| Flavour | 0.18% |
| Colour | 0.35% |
| Water | 7.53% |
| Total: | 100.00% |

### Example 17

### Delivery system for Antacids

The following delivery system was formulated to deliver about 500 mg of calcium carbonate and 400 mg each of aluminium and magnesium hydroxide in a 4.35 g dose. The product comprises over 30% w/w of functional ingredients, over 41% w/w of solvent and about 10% w/w of sugar syrups. The moisture content of final delivery system was approximately 16% by weight. The pH of the final product was 8.8 @ 20.8° C. and the a_{w} was 0.47.

| Ingredient | % by Weight |
|---|---|
| Glycerol | 39.96% |
| Propylene glycol | 1.31% |
| Calcium carbonate | 11.50% |
| Magnesium Hydroxide | 9.25% |
| Aluminium Hydroxide | 9.25% |
| 63 DE Corn syrup | 4.62% |
| High Fructose Corn Syrup | 5.42% |
| Gelatine | 3.90% |
| Pectin | 0.25% |
| Sweetening agents | 0.05% |
| Modified Starch | 1.00% |
| Flavour | 0.20% |
| Colour | 0.29% |
| Water | 13.00% |
| Total: | 100.00% |

The glycerol and propylene glycol were blended and the calcium carbonate, magnesium hydroxide and aluminium hydroxide dispersed therein and the blend warmed to 40-50° C. The sugar syrups were blended with the water and warmed to 60-70° C. The gelatine, pectin, sweetening agents and other dry ingredients were preblended and introduced into the syrup under shear. The antacid/solvent blend was then uniformly blended with the gelatine preparation. Flavour and colour were then added and the whole maintained between 40° C. and 55° C.

### Example 18

### Comparative Analysis of Different Processes for Preparing a Calcium Delivery System and Products of Same

This Example describes the results of a comparative study conducted to evaluate the differences between a process for the preparation of a calcium delivery system formulated according to one embodiment of the present invention (the “first process”) and a known process for the preparation of a calcium delivery system (as described in Yang et al., U.S. Pat. No. 5,928,664 [Sample 8, Table 1, and Examples 1 & 2 of Yang]; the “second process”), as well as the calcium delivery system products resulting from these two processes. The components used in the two processes to prepare the calcium delivery systems are shown in Table 8.

TABLE 8
Components Used to Prepare the Calcium Delivery Systems

| Process | | | % w/w Described in | % w/w Utilised |
|---|---|---|---|---|
| Used | Formulation | Ingredients | Yang | (adjusted as necessary)¹ |
| First | A | Gelatine | — | 4.52 |
| Process | | Glycerol | — | 38.5 |
| | | Water | — | 9.03 |
| | | Blended 63 DE glucose syrup and high | — | 15.68 |
| | | fructose syrup | | |
| | | Calcium carbonate | — | 30.18 |
| | | Starch (Miraquick ®) | — | 2.00 |
| | | Artificial Sweetener | — | 0.10 |
| | | Total: | — | 100 |
| Second | B | Glycerated Gelatine Matrix | | |
| Process | (Based on | Gelatine | 4.0 | 4.47 |
| | Yang, Sample | Glycerol | 12.83 | 14.34 |
| | 8, Table 1) | Initial Water | 12.83 | 14.34 |
| | | Initial weight of matrix: | 29.66 | 33.15 |
| | | Final weight of matrix²: | 19.12 | 21.38 |
| | | Final Delivery System | | |
| | | Glycerated gelatine matrix | 19.12 | 21.38 |
| | | Corn Sweetener Solids (36DE)³ | 38.19 | 42.69 |
| | | Calcium carbonate | 31.25 | 34.93 |
| | | Flavour & Artificial sweetener | 0.8995 | 1.01 |
| | | Total: | 89.46 | 100 |

¹In the second process the initial weight of ingredients is equal to 100%. Once the requisite amount of water had been driven off the glycerated gelatine matrix, a total of 89.46% of the initial 100% remains in the final product. In this Example, the amounts described in Yang were adjusted such that the total for the final product was 100%. The relative proportions of the ingredients, however, remains the same.
²Final weight of the glycerated gelatine matrix represents the weight after the requisite amount of water has been driven off by heating.
³As 36DE corn syrup solids (as used in Yang et al., U.S. Pat. No. 5,928,664) are not readily available, 36DE corn syrup solids for this Example were created by combining 57% 42DE corn syrup solids with 43% 28DE corn syrup solids.

In the first process, calcium carbonate was added to the glycerol and warmed to 65° C. The remaining ingredients (gelatine, water, glucose syrup/fructose syrup, starch and artificial sweetener) were combined and warmed to 65° C. The glycerol/calcium mix was combined with the gelatine mix with gentle stirring. The resultant solution was allowed to stand at 62-64° C. for 2-3 minutes with occasional mixing to ensure uniform temperature, and was then poured into moulds.

In the second process, the gelatine was hydrated according to standard methodology. Briefly, the gelatine and water were combined and allowed to stand at room temperature for ˜30 min. to allow hydration of the gelatine. The hydrated gelatine was then warmed over a water bath at about 50° C. to dissolve the gelatine. Once the gelatine had dissolved, the glycerol was added. Addition of the glycerol resulted in precipitation of the gelatine, which subsequently redissolved upon further heating. The solution was placed over a 95-98° C. water bath and heated for ˜3 h to remove the stipulated amount of water and provide a glycerated gelatine matrix with a water content of 12% (by weight of the matrix). The solid ingredients were combined and blended and then added to the glycerated gelatine matrix over the 95-98° C. water bath (internal temperature of matrix ˜90° C.) with mixing. Upon initiating addition of the solids, the solution rapidly became a pasty mass, which had to be maintained at high temperature in order to effect incorporation of additional solids. As the level of added solids increased, the mass could no longer be mixed manually as the pasty mass became a sticky solid that adhered to the walls of the mixing vessel. In order to effect complete incorporation of the solids and to distribute same throughout the solid mass, the sticky solid was turned out of the mixing vessel onto a hard surface and manually kneaded. The final sticky mass was allowed to cool slightly and then placed between two sheets of plastic wrap, covered and flattened to ˜1 cm in thickness, and finally refrigerated to permit easier sectioning into 1×2×2 cm pieces.

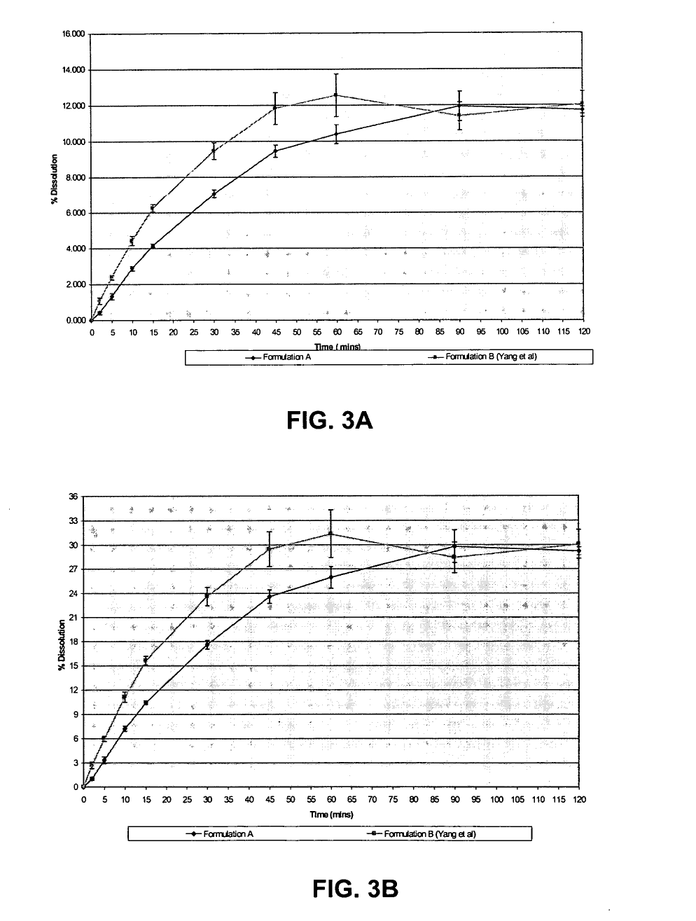

For the product produced by the first process (Formulation A), measurement of density was performed by pouring a pre-determined volume of the product into a tared measuring cylinder and recording the weight of the measured volume of product. For the product produced by the second process (Formulation B), the dough-like mass was cut into units measuring approximately 1 cm×2 cm×2 cm. Ten units were weighed and the density calculated from the average weight and volume. The melting point, dispersion of calcium and calcium release characteristics for the products of the two processes were determined using standard U.S. Pharmacopoeia (USP28—NF23) protocols. The release characteristics, as shown in FIG. 3, were assessed in standard simulated gastric fluid solution, without enzymes, according to USP protocols.

Results

The comparative data clearly illustrates differences between the processes and the products prepared by the two processes. For example, with respect to the processes, the first step in the second process requires the preparation of a “glycerated gelatine matrix” that requires admixing an aqueous solution of gelatine with glycerine (glycerol) and heating the resulting mixture in order to drive off water, thereby providing the glycerated gelatine matrix. This step is conducted prior to addition of the other components, including any active ingredient (in this instance, calcium) that is to be incorporated into the delivery system. In contrast, in the first process, the calcium is dispersed in the glycerine-containing solvent component prior to combining the solvent with any of the other components of the delivery system, including gelatine.

In addition, following the second process, removal of the requisite amount of water by heating the glycerol and gelatine mixture in a water bath at a temperature of 95-98° C. in order to achieve this final water content of 12% w/w required heating the mixture for over three hours and thus required considerable expenditure of energy in the form of heat. Addition of the solid ingredients to the glycerated gelatine matrix following the second process also rapidly resulted in the formation of a solid pasty mass that had to be maintained at the high temperature used to evaporate the water, in order to effect incorporation of the solids. Continued addition of solids increased the stickiness and doughy texture of the mass to the point that the mass could no longer be mixed within the mixing vessel, and it was subsequently turned out onto a solid surface and the remaining solids were incorporated by manual kneading. In order to effect incorporation of all the solids and distribute the solids throughout the mass, it was necessary to knead the mass over an extended period of time while gradually adding the remainder of the solid ingredients. This became increasingly difficult as the temperature of the mass decreased. Once the final product was obtained it was necessary to cut the mass into pieces of a size suitable for mastication or oral delivery of the calcium functional ingredient.

In contrast, the matrix prepared by the first process was formulated at the outset such that the final semi-solid gel delivery system would have the correct water content without the strict requirement to drive off water through the use of high temperatures for a prolonged period, thereby eliminating a time consuming and energy inefficient step necessary under the second process. Moreover, the product prepared by the first process remained flowable throughout the process, even though lower temperatures of about 65° C. were used, which allowed for quicker and less labour intensive incorporation, as well as thorough distribution, of all the components including the calcium active. The final non-solid product could be simply poured into moulds at the end of the process and allowed to cool and set, whereas this was not possible with the final solid product of the second process. The differences in preparation of the products of the two processes in a laboratory setting would also have implications for the application of the processes on a commercial scale; the second process requiring more specialised equipment to achieve complete mixing of all of the ingredients.

In addition, the final products made by the two processes differ substantially with respect to their density, melting point and calcium release characteristics, as shown in Table 9. The product of the second process (Formulation B) is denser than the product of the first process (Formulation A) and shows significantly different melting characteristics. Whereas Formulation A melts to a liquid at 43.18° C., Formulation B could not be melted to liquid form; the product simply softened even when the temperature was increased to 94° C. As is well known in the art, the temperature at which a solid or semi-solid melts is determined by the properties of the solid or semi-solid. Thus, the significant difference in melting temperatures between Formulation A and Formulation B is representative of one or more significant differences between the two products. The higher melting temperature and density of the product made by the second process is consistent with its design for the delivery of an active by way of prolonged mastication. By contrast, the characteristics of the product of the first process is more consistent with the objective of the flexible delivery of an active by way of gastro-intestinal ingestion in a variety of forms, including, for example, as a confectionery-like product unto itself, or as a filling in a wafer, or coating on a cookie. Once set, Formulation A had a texture and density analogous to a soft piece of liquorice or a jujube, whereas Formulation B was a “dough” type solid product that was pliable and chewable and more analogous to a piece of bubble gum.

As shown in Table 9, and in more detail in the dissolution profiles provided in FIG. 3, the calcium release characteristics of the two products were also significantly different. FIG. 3 shows in (A) the measured release of calcium, and in (B) the release of calcium, reported as calcium carbonate. Under gastric fluid solution, the release of calcium from the product of the second process can be seen to be much more rapid over the first 60 minutes than the release of calcium from the product of the first process. For example, at 30 and 60 minutes, the amount of calcium carbonate released from the product of the second process is 23.58 and 31.31% (w/w), respectively, whereas for the product of the first process, the amounts are 17.97 and 25.93% (w/w), respectively. The rate of release of an active ingredient from a delivery system is a function of the combined physical and chemical properties of the delivery system and thus the different release profiles demonstrated by the two products is also representative of the difference between the respective products.

TABLE 9
Characteristics of Calcium Delivery Systems Produced by First and Second Processes

| | Calculated Water | Density of | Melting Point | Release of Calcium from Final | |
|---|---|---|---|---|---|
| | Content of Final | Final Product | of Final | Product at 30 min. (% w/w of | Calcium Content of Final |
| Formulation | Product (% w/w) | (g/cc) | Product (° C.) | initial content ± Std. Dev.) | Product % w/w ± Std. Dev.² |
| A | 12.45 | 1.21 | 43.18 | 17.60 ± 0.53 | 28.56 ± 1.11 |
| (1^{st} process) | | | | | |
| B | 2.57 | 1.64¹ | >94³ | 23.58 ± 1.14 | 32.7 ± 1.02 |
| (2^{nd} process) | | | | | |

¹Calculated as an average of 10 units of product.
²Measured by atomic absorption spectrophotometry; values are an average for 5 units.
³Melting point of Formulation B could not be determined accurately. During preparation of the sample for melting point determination, the formulation softened, was elastic and very sticky. While attempting to take the melting point, the temperature was increased to 94° C. but no melting was observed, just a softening of the sample.

### Example 19

### Comparative Analysis of Different Processes for Preparing a Creatine Delivery and Products of Same

This Example describes the results of a comparative study conducted to evaluate the differences between a process for the preparation of a creatine delivery system formulated according to one embodiment of the present invention (the “first process”) and a known process for the preparation of a delivery systems (based on Sample 3, Table 1, and Examples 1 & 2 as described in Yang et al., U.S. Pat. No. 5,928,664; the “second process”), as well as the creatine delivery system products resulting from the two processes. The components used in the two processes to prepare the creatine delivery systems are shown in Table 10.

TABLE 10
Components of the Creatine Delivery Systems

| Process | | | % w/w | % w/w Utilised |
|---|---|---|---|---|
| Used | Formulation | Ingredients | Described in Yang | (adjusted as necessary)¹ |
| First | C | Gelatine | — | 4.79 |
| Process | | Glycerol | — | 28.08 |
| | | Propylene glycol | — | 3.42 |
| | | Water | — | 4.35 |
| | | Blended 63 DE glucose syrup | — | 30.55 |
| | | and high fructose syrup | | |
| | | Creatine | — | 24.0 |
| | | Potassium hydroxide | — | 0.3 |
| | | Potassium phosphate | — | 0.42 |
| | | Potassium citrate | — | 0.95 |
| | | Starch | — | 2.5 |
| | | Artificial sweetener | — | 0.04 |
| | | Flavour | — | 0.6 |
| | | Total: | — | 100 |
| Second | D | Glycerated Gelatine Matrix | | |
| Process | (Based on | Gelatine | 4.0 | 5.13 |
| | Yang, Sample | Glycerol | 12.83 | 16.46 |
| | 3, Table 1) | Initial Water | 20.0 | 16.46 |
| | | Initial weight of matrix: | 36.83 | 38.05 |
| | | Final weight of matrix²: | 19.12 | 24.53 |
| | | Final Delivery System | | |
| | | Glycerated gelatine matrix | 19.12 | 24.53 |
| | | Corn Sweetener Solids (36DE)³ | 26.69 | 50.32 |
| | | Active ingredient⁴ | 31.25 | 24.0 |
| | | | (CaCO₃) | (creatine) |
| | | Flavour | 0.7 | 0.9 |
| | | Artificial sweetener | 0.1995 | 0.25 |
| | | Total: | 77.95 | 100 |

¹In the second process the initial weight of ingredients is equal to 95.67%. Once the requisite amount of water had been driven off the glycerated gelatine matrix, a total of 77.95% of the initial 95.67% remains in the final product. In this Example, the amounts described in Yang were adjusted such that the total for the final product was 100%. The relative proportions of the ingredients, however, remains the same except where indicated.
²Final weight of the glycerated gelatine matrix represents the weight after the requisite amount of water has been driven off by heating.
³See footnote 3 to Table 8.
⁴CaCO₃ in Sample 3, Table 1 of the second process was substituted with creatine and the amount of corn syrup solids was adjusted in order to keep the total % of solid ingredients included in the formulation the same as that for Sample 3.

In the first process, glycerol and propylene glycol were first combined with the potassium hydroxide and then the creatine was added and stirred in. This creatine blend was warmed to 50-52° C. Separately, the water and blended syrups were combined. The buffer salts, starch and gelatine were blended into the syrups/water mixture and the blend was warmed to 60-62° C. The two blends were combined at a temperature of 50-52° C. and the resulting fluid matrix was poured into moulds.

In the second process, a glycerated gelatine matrix with a water content of 12% (by weight of the matrix) was first prepared as described in Example 18. The solid ingredients were next combined and blended. The blended solids were then added to the glycerated gelatin matrix over the 95-98° C. water bath (internal temperature of matrix ˜85-90° C.) with mixing. As was the case with the preparation of the calcium delivery system using this method (as described in Example 18), addition of the solids resulted in a pasty mass, which had to be maintained at high temperature in order to effect incorporation of additional solids. Increased levels of added solids produced a sticky solid that adhered to the walls of the mixing vessel, which had to be turned out of the mixing vessel onto a hard surface and manually kneaded in order to effect complete incorporation and distribution of the solids. The final sticky mass was allowed to cool slightly and then placed between two sheets of plastic wrap, covered and flattened to ˜1 cm in thickness, and finally refrigerated to permit easier sectioning into 1×2×2 cm pieces.

The density of the two creatine delivery system products produced by the above methods was measured by standard calculation as described in Example 18. Melting point and pH were determined using standard U.S. Pharmacopoeia (USP28—NF23) protocols. Creatine and creatinine content were measured by reverse-phase high performance liquid chromatography (HPLC).

Results

The comparative data clearly illustrates the differences between the two processes and the creatine delivery system products prepared by these processes. In particular, when preparing the creatine product following the second process, the same difficulties were encountered as during the preparation of the calcium product using the second process as described in Example 18. The comparative data thus indicates that the first process for the preparation of a creatine delivery system is a more versatile and commercially attractive process to exploit from a manufacturing and packaging point of view.

With respect to the creatine products produced by the two processes, the results shown in Table 11 demonstrate that the final products made by the two processes differ substantially in density, melting point, creatine dispersion and pH. As was observed for the products in Example 18, the creatine product of the second process (Formulation D) is denser than the creatine product of first process (Formulation C) and shows significantly different melting characteristics. Formulation C melted to a liquid at 38.8° C., whereas Formulation D did not melt to produce a liquid, but rather softened and stretched to the point that, at 64.8° C., the product extended from the end of the thermometer to the base of the experimental vessel and rendered further testing impossible. Again, the significant difference in melting temperatures observed is representative of one or more significant differences between the two products. Also, as with the calcium delivery system of Example 18, the first process for preparing the creatine delivery system resulted in a final product having a texture and density analogous to a soft piece of liquorice or a jujube, while that of the second, known process was more analogous to a piece of bubble gum.

As also shown in Table 11, the distribution of creatine within the product of the first process was considerably more uniform than that within the creatine product of the second process. The average creatine content for 5 samples of Formulation C was 25.302+0.080% w/w of creatine, with a maximum difference between samples of just 0.17%. The average creatine content for 5 samples of Formulation D was 25.066+0.511% w/w of creatine, with a maximum difference between samples of 1.28%. The markedly consistent distribution of the creatine in Formulation C is a direct result of the process used to produce this product, which allows for thorough mixing of the creatine/solvent solution into the remaining ingredients in a liquid phase.

TABLE 11
Characteristics of Creatine Delivery Systems Produced by First and Second Processes

| | Calculated | | | | | |
|---|---|---|---|---|---|---|
| | Water | | | | | |
| | Content of | Density of | | Maximum Difference in Creatine | | |
| | Final | Final | Melting Point | Content between Samples % w/w | | Creatinine Content |
| | Product | Product | of Final | (Creatine Content of Final Product | pH of Final | of Final Product |
| Formulation | (% w/w) | (g/cc) | Product (° C.) | % w/w ± Std. Dev.)² | Product | (% w/w ± Std. Dev.) |
| C | 11.17 | 1.22 | 38.8 | 0.17 | 8.79 | 0.338 ± 0.277 |
| (1^{st} process) | | | | (25.302 ± 0.080) | | |
| D | 2.94 | 1.64¹ | Softens at | 1.28 | 6.93 | 0.316 ± 0.027 |
| (2^{nd} process) | | | 64.8³ | (25.066 ± 0.511) | | |

¹Calculated as an average of 10 units of product.
²Average of 5 units.
³Formulation D did not melt completely, but at the given temperature had softened and stretched to the extent that further assessment became impracticable.

### Example 20

### Other Nutritional Supplements Delivery Systems

Delivery systems incorporating the nutritional supplements listed in Table 12, alone and in various combinations, were prepared according to the process described below, which includes the step of dispersing a nutritional supplement in the solvent system. All the delivery systems were formulated to a final moisture content between 10% and 30% w/w and a water activity of less than 0.7 and remained flowable at a temperature of about 45° C. Specific formulations including the nutritional supplements marked with an asterisk (*) in Table 11 are described in Examples 1 to 6 or 10.

Each delivery system was prepared using the following components in amounts within the stipulated ranges:

| | |
|---|---|
| Glycerol and optionally propylene glycol: | 5%-35% w/w |
| Sugars, sugar alcohols and/or sugar syrups: | 20%-60% w/w |
| Modified starch and optionally other carbohydrates: | 1%-15% w/w |
| Gelatine and optionally pectin or gellan: | 0.1%-7% w/w |
| Nutritional supplement(s): | less than |
| | 25% w/w total |

Process

Glycerol and propylene glycol were blended together to provide a solvent system. Optionally, one or more pH modifying agents were added to the solvent system and blended in. The one or more nutritional supplements were mixed into the solvent system, together or in a stepwise manner, and the solvent system was warmed to a temperature less than 70° C. Separately, the sugars, sugar alcohols and/or sugar syrups were blended together. Starch and gelatine and optionally pectin or gellan were pre-blended, and pH modifying agents and artificial sweeteners were included as required. The sugar and starch mixtures were combined and blended to provide a sugar/starch blend. Water was added at this point if necessary such that the final product had the desired final moisture content. The sugar/starch blend was heated to about 70° C. and subsequently cooled down to about 50° C. The sugar/starch blend and the solvent system were blended together. Flavours and colours were blended into the final mixture, as desired, and the final flowable mixture was moulded and allowed to cool to provide the delivery system.

The range and diversity of nutritional supplements that were successfully incorporated into the delivery system using the above process thus demonstrates the flexibility and broad applicability of both the process and the delivery system.

TABLE 12
Exemplary Nutritional Supplements Incorporated into Delivery Systems

| | In Combination with |
|---|---|
| | Other Nutritional |
| Nutritional Supplement Incorporated | Supplements |
| into Delivery System | (Y/N) |
| Aloe extract | Y |
| Arginine* | Y |
| Astaxanthin | Y |
| Black tea extract | Y |
| Caffeine* | Y |
| Calcium carbonate | Y |
| L-Carnitine | Y |
| L-Carnosine | Y |
| β-Carotene | Y |
| Chondroitin sulfate | Y |
| Chromium chelate* | Y |
| Citrulline | Y |
| Citrus aurantium extract* | Y |
| Citrus derived antioxidants | N |
| Co-enzyme Q₁₀ (including liposomal CoQ₁₀) | Y |
| Conjugated linoleic acid (CLA)* | Y |
| Copper gluconate | Y |
| Creatine magnesium chelate | Y |
| Creatine monohydrate* | Y |
| Dimethylglycine* | Y |
| Epimedium extract | Y |
| Fiber (soy source) | N |
| Fructo-oligosaccharides | Y |
| Fructus lycii (Gou Qi Zi) extract | Y |
| Fruit extract (pomegranate, cherry, apple skin) | Y |
| Ginseng | Y |
| α-Glyceryl phoshorylcholine | Y |
| Glucosamine salts | Y |
| Glutamine | N |
| Green tea extract | Y |
| Guarana extract | Y |
| Gymnema sylvestre extract | Y |
| Histidine | Y |
| Hydroxycitric acid | Y |
| 5-Hydroxytryptophan (5-HTP) | Y |
| Inulin* | Y |
| Isoleucine | Y |
| Isoflavones (clover or soy) | Y |
| Leucine | Y |
| Lecithin* | Y |
| α-Lipoic acid | Y |
| Lysine | Y |
| Magnesium glycyl glutamine | Y |
| Magnesium phosphate | Y |
| Magnesium sulphate | Y |
| Ma Huang extract (ephedra) | Y |
| Manganese sulphate | Y |
| Methionine | Y |
| Methyl sulfonyl methane (MSM) | Y |
| Microcrystalline hydroxyapatite complex (MCHC) | Y |
| Octacosanol | Y |
| Phenylalanine | Y |
| Potassium bicarbonate | Y |
| Potassium phosphate | Y |
| Quercetin | Y |
| Rhodiola rosea extract* | Y |
| Seabuckthorn extract* | Y |
| Selenium selenite | Y |
| Sodium citrate | Y |
| Sodium phosphate | Y |
| Taurine | Y |
| Threonine | Y |
| Tribulus terrestris extract | Y |
| Trimethylglycine (betaine) | Y |
| Tyrosine | Y |
| Valine | Y |
| Vitamin B₁ (thiamin) | Y |
| Vitamin B₂ (riboflavin) | Y |
| Vitamin B₃ (niacin) | Y |
| Vitamin B₅ (calcium panthothenate) | Y |
| Vitamin B₆ (pyroxidine) | Y |
| Vitamin B₁₂ (cyanocobalamine) | Y |
| Vitamin C (ascorbic acid)* | Y |
| Vitamin D | Y |
| Vitamin E (mixed tocopherols)* | Y |
| Yerba mate (Ilex paraguariensis extract) | Y |
| Zinc arginine chelate | N |
| Zinc gluconate | Y |

### Example 21

### Delivery Systems Comprising Nutritional Supplement Combinations

Delivery systems incorporating exemplary combinations of nutritional supplements as shown in Table 13 were prepared according to the process described in Example 20. For all combinations, at least one nutritional supplement was added to the solvent component (i.e. glycerol and propylene glycol) as described in Example 20. However, one or more nutritional supplements could optionally be combined with the sugar/starch mixture, where the additional nutritional supplement(s) is amenable to incorporation by such a step.

For example, when the nutritional supplement caffeine was included in the delivery system in the form of pure caffeine powder, the caffeine powder was first dissolved in a fixed weight of water with heating to 80° C. Water was added back as necessary, at room temperature, to correct for water loss during heating. The caffeine solution was then combined with the blended sugars, sugar alcohols and/or sugar syrups, which were at room temperature. The other nutritional supplement(s) included in the delivery system in combination with the caffeine were added to the glycerol/propylene glycol solvent component. When caffeine was used in a coated form (for example, nano-encapsulated or microencapsulated caffeine), however, it was added into the solvent system.

Similarly, when the nutritional supplement glucosamine was included in the delivery system, the glucosamine was combined with the blended sugars, sugar alcohols and/or sugar syrups, which were at room temperature. The other nutritional supplement(s) included in the delivery system in combination with the glucosamine were added to the glycerol/propylene glycol solvent component.

The final delivery systems all had a final moisture content between 10% and 30% w/w, a water activity of less than 0.7 and remained flowable at a temperature of about 45° C. Each of the blends represented by a footnote (1-5) are themselves representative of combinations of more than one nutritional supplement.

The delivery systems shown in Table 13 demonstrate the flexibility of the process for incorporating combinations of compounds having different chemical and physical properties. By way of example, product “A” in Table 13 includes a combination of a lipophilic compound (vitamin E), water-soluble compounds (caffeine and phosphate salts) and botanical extracts, which extracts contain complex mixtures of compounds.

TABLE 13
Delivery Systems Comprising Exemplary Combinations of Nutritional Supplements

| | | No. of Nutritional | | Amount of each | Total Amount of Combined |
|---|---|---|---|---|---|
| | Weight of | Supplements | Nutritional | Nutritional Supplement | Nutritional Supplements Included in |
| Product | Product/g | Incorporated | Supplement | Included in Product/mg | Product/mg (% w/w) |
| A | 13 | 8 | Caffeine | 100 | 1965.0 |
| | | | Phosphate blend #1¹ | 600 | (15.1%) |
| | | | Herbal blend² | 1000 | |
| | | | Sodium citrate | 250 | |
| | | | Vitamin E | 15 | |
| B | 13 | 9 | Caffeine | 200 | 1469.8 |
| | | | L-carnitine | 500 | (11.3%) |
| | | | Taurine | 250 | |
| | | | Tyrosine | 250 | |
| | | | Vitamin B₁ | 1.2 | |
| | | | Vitamin B₂ | 1.3 | |
| | | | Vitamin B₃ | 16 | |
| | | | Vitamin B₆ | 1.3 | |
| | | | Yerba mate | 250 | |
| C | 12 | 10 | L-carnitine | 250 | 1944.6 |
| | | | Phosphate blend #2³ | 600 | (16.2%) |
| | | | Amino acid blend #1⁴ | 1078 | |
| | | | Manganese sulphate | 1.0 | |
| | | | Selenium selenite | 0.62 | |
| | | | Zinc gluconate | 15 | |
| D | 13 | 9 | Caffeine | 100 | 1480.0 |
| | | | L-carnitine | 500 | (11.4%) |
| | | | Herbal blend² | 150 | |
| | | | Quercetin | 30 | |
| | | | Taurine | 250 | |
| | | | Tyrosine | 250 | |
| | | | Yerba mate | 200 | |
| E | 11.5 | 9 | L-carnosine | 100 | 2100.0 |
| | | | Amino acid blend #2⁵ | 1750 | (18.3%) |
| | | | Rhodiola rosea extract | 250 | |
| F | 12 | 6 | Caffeine | 30.6 | 1176.6 |
| | | | Calcium carbonate | 46 | (9.8%) |
| | | | Citrus aurantium extract | 61 | |
| | | | Conjugated linoleic acid | 957 | |
| | | | Inulin | 76.6 | |
| | | | Vitamin E | 5.4 | |

¹Blend of two different phosphate salts
²Blend of three different herbal extracts
³Blend of three different phosphate salts
⁴Blend of three different amino acids
⁵Blend of seven different amino acids

### Example 22

### Delivery System for Calcium and Vitamin E

The following delivery system was formulated to deliver calcium and vitamin E in a 5 g final product. The delivery system comprises greater than 48% w/w of solvent and less than 18% w/w sugar syrups. The delivery system was prepared by the process according to Example 20. The final product had a pH of 7.19 (26.1° C., and an a_{w} of 0.55.

| Ingredient | % by Weight |
|---|---|
| Total solvent (glycerol and propylene glycol) | 48.81% |
| Calcium | 15.0% |
| Vitamin E | 0.6% |
| Blend of corn syrup and HFCS | 17.44% |
| Water | 11.37% |
| Modified starch | 1.54% |
| Gelatine | 4.56% |
| Flavours & Colours | 0.37% |
| Pectin | 0.25% |
| Sucralose/Ace K/sorbitol | 0.06% |
| Total: | 100.0% |

### Example 23

### Delivery System for a Calcium-Containing Antacid

The following delivery system was formulated to deliver calcium, aluminium hydroxide and magnesium hydroxide in a 6 g final product. The delivery system comprises greater than 47% w/w of solvent and about 11% w/w sugar syrups. The delivery system was prepared by the process according to Example 20. The final product had a pH of 8.93 @ 26.1° C., and an a_{w} of 0.52.

| Ingredient | % by Weight |
|---|---|
| Total solvent (glycerol and propylene glycol) | 47.41% |
| Calcium | 8.33% |
| Aluminium hydroxide | 6.67% |
| Magnesium hydroxide | 6.67% |
| Blend of corn syrup and HFCS | 11.04% |
| Water | 13.33% |
| Modified starch | 1.69% |
| Gelatine | 4.06% |
| Pectin | 0.25% |
| Flavours & Colours | 0.49% |
| Sucralose/Ace K/sorbitol | 0.06% |
| Total: | 100.00% |

### Example 24

### Delivery System for an Antacid

The following delivery system was formulated to deliver aluminium hydroxide, magnesium hydroxide and simethicone in a 9 g final product. The delivery system comprises greater than 41% w/w of solvent, 15% w/w sugar syrups, less than 1% w/w modified starch and about 28% w/w functional ingredients. The delivery system was prepared by the process according to Example 20. The final product had a pH of 9.33 @ 23.6° C., and an a_{w} of 0.53.

| Ingredient | % by Weight |
|---|---|
| Total solvent (glycerol and propylene glycol) | 41.39% |
| Aluminium hydroxide | 13.34% |
| Magnesium hydroxide | 13.33% |
| Simethicone | 1.34% |
| Blend of corn syrup and HFCS | 15.00% |
| Water | 11.54% |
| Modified starch | 0.60% |
| Gelatine | 2.80% |
| Flavours & Colours | 0.52% |
| Pectin | 0.10% |
| Sucralose/Ace K | 0.04% |
| Total: | 100.00% |

### Example 25

### Delivery System for Cough Suppressant

The following drug delivery system was formulated to deliver the drugs guaifenesin and dextromethorphan in a 3 g final product. The delivery system comprises greater than 37% w/w of solvent and greater than 10% w/w of hydrocolloids (i.e. gelatine and pectin).

The delivery system was prepared by the process according to Example 20, with the following modification. The guaifenesin and dextromethorphan were mixed into the solvent system, together or in a stepwise manner, and the solvent system was warmed to 75-80° C. for a complete dissolution. The delivery system could also be successfully prepared by warming the solvent system to less than 70° C., as described in Example 20. The delivery system prepared by this latter method had a slightly gritty texture and a higher bitterness level due to the incomplete dissolution of the guaifenesin, which reduced the palatability of the final product. The final product had a pH of 6.44® 24.3° C., and an aw of 0.56.

| Ingredient | % by Weight |
|---|---|
| Total solvent (glycerol and propylene glycol) | 37.42% |
| Guaifenesin | 6.67% |
| Dextromethorphan | 0.66% |
| Blend of corn syrup and HFCS | 31.05% |
| Water | 11.04% |
| Modified starch | 1.59% |
| Gelatine | 10.35% |
| Flavours & Colours | 0.81% |
| Sucralose/Ace K/sorbitol | 0.06% |
| Pectin | 0.35% |
| Total: | 100.00% |

### Example 26

### Delivery System for Citrus Extract

The following delivery system was formulated to deliver a citrus extract in a 5 g final product. The delivery system was prepared by the process according to Example 16. The delivery system comprises greater than 41% w/w of solvent. The final product had a pH of 6.27® 22.2° C., an a_{w} of 0.52 and a final moisture content of 16.1%.

| Ingredient | % by Weight |
|---|---|
| Glycerol | 39.64% |
| Propylene glycol | 2.09% |
| Citrus extract | 2.64% |
| Blend of corn syrup and HFCS | 41.34% |
| Water | 4.11% |
| Modified Potato Starch | 2.19% |
| Gelatine | 6.58% |
| Flavours & colours | 0.81% |
| Pectin | 0.32% |
| Sucralose/Ace K/sorbitol | 0.28% |
| Total: | 100.00% |

### Example 27

### Delivery System for Vitamin C and Caffeine

The following delivery system was formulated to deliver vitamin C and caffeine in a 5 g final product. The delivery system was prepared by the process according to Example 16. The delivery system comprises greater than 37% w/w of solvent. The final product had a pH of 6.62 @ 22.1° C., an a_{w} of 0.48, and a final moisture content of 15.3%.

| Ingredient | % by Weight |
|---|---|
| Glycerol | 35.21% |
| Propylene glycol | 2.31% |
| Vitamin C | 8.33% |
| Caffeine | 0.83% |
| Blend of corn syrup and HFCS | 41.09% |
| Water | 6.01% |
| Modified Potato Starch | 1.50% |
| Gelatine | 6.00% |
| Flavours & colours | 0.65% |
| Pectin | 0.30% |
| Sucralose/Ace K/sorbitol | 0.14% |
| Total: | 100.00% |

### Example 28

### Delivery System for Arginine, Creatine and Other Functional Ingredients

The following delivery system was formulated to deliver arginine, creatine, a blend of amino acids, vitamin B3 and caffeine (providing a total functional ingredient content of 21.34%) in a 5 g final product. The delivery system was prepared by the process according to Example 16. The delivery system comprises greater than 36% w/w of solvent, and a modified cellulose (methylcellulose). The final product had a pH of 9.21 @ 22.4° C., an a_{w} of 0.37 and a final moisture content of 15.7%.

| Ingredient | % by Weight |
|---|---|
| Glycerol | 33.83% |
| Propylene glycol | 2.82% |
| Arginine | 11.54% |
| Creatine | 7.69% |
| Taurine | 0.77% |
| Tyrosine | 0.39% |
| Valine | 0.12% |
| Leucine | 0.08% |
| Isoleucine | 0.08% |
| Histidine | 0.08% |
| Methionine | 0.04% |
| Vitamin B3 | 0.16% |
| Caffeine | 0.39% |
| Blend of corn syrup and HFCS | 21.97% |
| Water | 7.66% |
| Potassium citrate) | 0.94% |
| Modified Potato Starch | 1.99% |
| Gelatine | 4.86% |
| Phosphoric acid | 1.50% |
| Methylcellulose | 1.00% |
| Flavours & colours | 1.64% |
| Pectin | 0.25% |
| Sucralose/Ace K/sorbitol | 0.20% |
| Total: | 100.00% |

### Example 29

### Delivery System for Fibre

The following delivery system was formulated to deliver methylcellulose (as a source of fibre) in a 5 g final product. The delivery system was prepared by the process according to Example 16. The delivery system comprises greater than 38% w/w of solvent and greater than 7% hydrocolloid (gelatine+pectin). The final product had a pH of 6.76 @ 23.3° C., an a_{w} of 0.58, and a final moisture content of 18.7%. Ingredient % by Weight

| Ingredient | % by Weight |
|---|---|
| Glycerol | 34.67% |
| Propylene glycol | 4.24% |
| Methylcellulose (fibre source) | 7.69% |
| Blend of corn syrup and HFCS | 30.21% |
| Water | 11.85% |
| Modified potato starch | 3.10% |
| Gelatine | 7.51% |
| Flavours & colours | 0.39% |
| Pectin | 0.31% |
| Sucralose/Ace K/sorbitol | 0.03% |
| Total: | 100.00% |

### Example 30

### Delivery System for Caffeine

The following delivery system was formulated to deliver caffeine in a 5 g final product. The delivery system was prepared by the process according to Example 16. The delivery system comprises greater than 47% w/w of solvent. The final product had a pH of 7.82 @ 22.4° C., an a_{w} of 0.56, and a final moisture content of 17.8%.

| Ingredient | % by Weight |
|---|---|
| Glycerol | 45.02% |
| Propylene glycol | 2.32% |
| Caffeine | 2.39% |
| Blend of corn syrup and HFCS | 32.08% |
| Water | 10.67% |
| Modified potato starch | 1.51% |
| Gelatine | 5.10% |
| Flavours & colours | 0.55% |
| Pectin | 0.30% |
| Sucralose/Ace K/sorbitol | 0.06% |
| Total: | 100.00% |

The disclosure of all patents, publications, including published patent applications, and database entries referenced in this specification are specifically incorporated by reference in their entirety to the same extent as if each such individual patent, publication, and database entry were specifically and individually indicated to be incorporated by reference.

Although the invention has been described with reference to certain specific embodiments, various modifications thereof will be apparent to those skilled in the art without departing from the spirit and scope of the invention. All such modifications as would be apparent to one skilled in the art are intended to be included within the scope of the following claims.

What is claimed is:

1. A semi-solid oral gel delivery system for functional ingredients comprising an effective amount of one or more functional ingredients substantially uniformly dispersed throughout a semi-solid matrix, said semi-solid matrix formulated from:
(a) between about 8% and about 60% by weight of one or more sugars, sugar syrups, or sugar alcohols, or a combination thereof;
(b) between about 0.5% and about 15% by weight of a carbohydrate component comprising one or more starches or modified starches;
(c) between about 0.1% to about 15% by weight of one or more hydrocolloids, and
(d) between about 5% and about 50% by weight of a solvent component comprising glycerol,
wherein said delivery system has a final moisture content of between about 10% and about 40% by weight, a water activity of less than about 0.9, and a melting temperature between about 30° C. and about 60° C.

2. The semi-solid oral gel delivery system according to claim 1, wherein said carbohydrate component further comprises a vegetable gum, a modified vegetable gum, a cellulose, a modified cellulose, or a combination thereof.

3. The semi-solid oral gel delivery system according to claim 1, wherein said solvent component further comprises a lower alkyl ester derivative of glycerol, propylene glycol, a short chain polyalkylene glycol, or a combination thereof.

4. The semi-solid oral gel delivery system according to claim 1, wherein said delivery system has a final pH between about 2.5 to about 10.0.

5. The semi-solid oral gel delivery system according to claim 1, wherein said delivery system has a final moisture content of between about 10% and about 30% by weight, a water activity of less than about 0.7.

6. The semi-solid oral gel delivery system according to claim 1, wherein said one or more sugars, sugar syrups, or sugar alcohols, or combination thereof, is one or more sugar syrups.

7. The semi-solid oral gel delivery system according to claim 6, wherein said one or more sugar syrups comprise one or more corn syrups.

8. The semi-solid oral gel delivery system according to claim 1, wherein said one or more hydrocolloids comprise gelatine.

9. The semi-solid oral gel delivery system according to claim 1, wherein said one or more hydrocolloids comprise gelatine and gellan or pectin.

10. The semi-solid oral gel delivery system according to claim 9, wherein said hydrocolloid component comprises gelatine and pectin in a ratio between about 15:1 and about 35:1.

11. The semi-solid oral gel delivery system according to claim 3, wherein said solvent component comprises glycerol and propylene glycol.

12. The semi-solid oral gel delivery system according to claim 1, wherein said one or more functional ingredients are one or more drugs suitable for oral administration, one or more nutritional supplements, or a combination thereof.

13. The semi-solid oral gel delivery system according to claim 1, wherein said one or more functional ingredients are one or more drugs suitable for oral administration.

14. The semi-solid oral gel delivery system according to claim 13, wherein said one or more drugs suitable for oral administration are selected from the group of: an acid-lipid agent, an alkaloid, an anabolic drug, an antacid, an anti-asthmatic, an anti-anginal drug, an anti-arrhythmic, an antibiotic, an antibody, an anti-cholesterolemic, an anti-coagulant, an anti-convulsant, an anti-diarrhoeal, an anti-emetic, an anti-fungal, an antigen, an anti-histamine, an anti-hypertensive drug, an anti-inflammatory drug, an anti-manic, an anti-migraine drug, an anti-nauseant, an anti-obesity drug, an anti-psychotic, an anti-pyretic, an anti-spasmodic agent, an anti-thyroid preparation, an anti-thrombotic drug, an anti-tumour compound, an anti-tussive, an anti-uricemic drug, an anti-viral, a cerebral dilator, a cholesterol lowering drug, a contrast agent, a coronary dilator, a decongestant, a diuretic, an erythropoietic drug, an expectorant, a gastrointestinal sedative, a hormone, a hyperglycaemic agent, a hypnotic, a hypoglycaemic agent, a laxative, a local anaesthetic, a mucolytic, a neuromuscular drug, a peripheral vasodilator, a prokinetic drug, a proton pump inhibitor, a psychotropic, a sedative, a stimulant, a thyroid preparation, a tranquilliser, a uterine relaxant, a vasoconstrictor, a vasodilator and a vasopressor.

15. The semi-solid oral gel delivery system according to claim 1, wherein said one or more functional ingredients are one or more nutritional supplements.

16. The semi-solid oral gel delivery system according to claim 15, wherein said one or more nutritional supplements are selected from the group of: an antioxidant, an amino acid, an amino acid derivative, a bee product, a botanical extract, a choline source, a co-enzyme, a co-factor, a dipeptide, an enzyme, a fatty acid, a fibre, a herbal extract, a hormone, a joint health nutrient, a macro-nutrient, a metabolic intermediate, a micro-nutrient, a mineral, a mineral salt, an oxygenator, a phospholipid, a phytochemical, a prebiotic, a probiotic bacterium, a protein, and a vitamin.

17. The semi-solid oral gel delivery system according to claim 1, wherein said delivery system comprises between about 8% and about 55% by weight of said one or more sugars, sugar syrups, or sugar alcohols, or combination thereof.

18. The semi-solid oral gel delivery system according to claim 1, wherein said delivery system comprises between about 0.5% and about 10% by weight of said carbohydrate component.

19. The semi-solid oral gel delivery system according to claim 1, wherein said delivery system comprises between about 0.5% to about 12% by weight of said one or more hydrocolloids.

20. The semi-solid oral gel delivery system according to claim 1, wherein said delivery system comprises between about 5% and about 48% by weight of said solvent component.

21. A semi-solid oral gel delivery system for functional ingredients comprising an effective amount of one or more functional ingredients substantially uniformly dispersed throughout a semi-solid matrix formulated from between about 8% and about 60% byweight of one or more sugars, sugar syrups, or sugar alcohols, or a combination thereof; between about 0.5% and about 15% by weight of a carbohydrate component comprising one or more starches or modified starches; between about 0.1% to about 15% by weight of one or more hydrocolloids, and between about 5% and about 50% by weight of a solvent component comprising glycerol, wherein said delivery system has a final moisture content of between about 10% and about 40% by weight, a water activity of less than about 0.9, and a melting temperature between about 30° C. and about 60° C. and is prepared by a process comprising the steps of:
(a) dispersing said effective amount of the one or more functional ingredients in said solvent component below a temperature of 100° C. to provide a solvent mixture;
(b) blending said solvent mixture at a temperature between about 50° C. and 80° C. with a blend comprising said one or more sugars, sugar alcohols or sugar syrups, or combination thereof; said carbohydrate component; said one or more hydrocolloids, and optionally water, to provide a flowable matrix in which said one or more functional ingredients are substantially uniformly dispersed, and
(c) moulding said matrix and allowing it to cool to provide said semi-solid oral gel delivery system.

22. The semi-solid oral gel delivery system according to claim 21, wherein said carbohydrate component further comprises a vegetable gum, a modified vegetable gum, a cellulose, a modified cellulose, or a combination thereof.

23. The semi-solid oral gel delivery system according to claim 21, wherein said solvent component further comprises a lower alkyl ester derivative of glycerol, propylene glycol, a short chain polyalkylene glycol, or a combination thereof.

24. The semi-solid oral gel delivery system according to claim 21, wherein said delivery system has a final pH between about 2.5 to about 10.0.

25. The semi-solid oral gel delivery system according to claim 21, wherein said delivery system has a final moisture content of between about 10% and about 30% by weight, a water activity of less than about 0.7.

26. The semi-solid oral gel delivery system according to claim 21, wherein said one or more sugars, sugar syrups, or sugar alcohols, or combination thereof, is one or more sugar syrups.

27. The semi-solid oral gel delivery system according to claim 26, wherein said one or more sugar syrups comprise one or more corn syrups.

28. The semi-solid oral gel delivery system according to claim 21, wherein said one or more hydrocolloids comprise gelatine.

29. The semi-solid oral gel delivery system according to claim 21, wherein said one or more hydrocolloids comprise gelatine and gellan or pectin.

30. The semi-solid oral gel delivery system according to claim 29, wherein said hydrocolloid component comprises gelatine and pectin in a ratio between about 15:1 and about 35:1.

31. The semi-solid oral gel delivery system according to claim 23, wherein said solvent component comprises glycerol and propylene glycol.

32. The semi-solid oral gel delivery system according to claim 21, wherein said one or more functional ingredients are one or more drugs suitable for oral administration, one or more nutritional supplements, or a combination thereof.

33. The semi-solid oral gel delivery system according to claim 21, wherein said one or more functional ingredients are one or more drugs suitable for oral administration.

34. The semi-solid oral gel delivery system according to claim 33, wherein said one or more drugs suitable for oral administration are selected from the group of: an acid-lipid agent, an alkaloid, an anabolic drug, an antacid, an anti-asthmatic, an anti-anginal drug, an anti-arrhythmic, an antibiotic, an antibody, an anti-cholesterolemic, an anti-coagulant, an anti-convulsant, an anti-diarrhoeal, an anti-emetic, an anti-fungal, an antigen, an anti-histamine, an anti-hypertensive drug, an anti-inflammatory drug, an anti-manic, an anti-migraine drug, an anti-nauseant, an anti-obesity drug, an anti-psychotic, an anti-pyretic, an anti-spasmodic agent, an anti-thyroid preparation, an anti-thrombotic drug, an anti-tumour compound, an anti-tussive, an anti-uricemic drug, an anti-viral, a cerebral dilator, a cholesterol lowering drug, a contrast agent, a coronary dilator, a decongestant, a diuretic, an erythropoietic drug, an expectorant, a gastrointestinal sedative, a hormone, a hyperglycaemic agent, a hypoglycaemic agent, a hypnotic, a laxative, a local anaesthetic, a mucolytic, a neuromuscular drug, a peripheral vasodilator, a prokinetic drug, a proton pump inhibitor, a psychotropic, a sedative, a stimulant, a thyroid preparation, a tranquilliser, a uterine relaxant, a vasoconstrictor, a vasodilator and a vasopressor.

35. The semi-solid oral gel delivery system according to claim 21, wherein said one or more functional ingredients are one or more nutritional supplements.

36. The semi-solid oral gel delivery system according to claim 35, wherein said one or more nutritional supplements are selected from the group of: an antioxidant, an amino acid, an amino acid derivative, bee pollen, bee propolis, a botanical extract, a choline source, a co-enzyme, a co-factor, a dipeptide, an enzyme, a fatty acid, a fibre, a herbal extract, a hormone, a joint health nutrient, a macro-nutrient, a metabolic intermediate, a micro-nutrient, a mineral, a mineral salt, an oxygenator, a phospholipid, a phytochemical, a prebiotic, a probiotic bacterium, a protein, Royal jelly and a vitamin.

37. The semi-solid oral gel delivery system according to claim 21, wherein said delivery system comprises between about 8% and about 55% by weight of said one or more sugars, sugar syrups, or sugar alcohols, or combination thereof.

38. The semi-solid oral gel delivery system according to claim 21, wherein said delivery system comprises between about 0.5% and about 10% by weight of said carbohydrate component.

39. The semi-solid oral gel delivery system according to claim 21, wherein said delivery system comprises between about 0.5% to about 12% by weight of said one or more hydrocolloids.

40. The semi-solid oral gel delivery system according to claim 21, wherein said delivery system comprises between about 5% and about 48% by weight of said solvent component.
